(19) United States
(12) Patent Application Publication
(10) Pub. No.: US 2026/0117240 A1
(43) Pub. Date: Apr. 30, 2026

(54) Enhancing Pro-vitamin A Biofortification of Fruit

(71) Applicant: UNIVERSITY OF FLORIDA RESEARCH FOUNDATION, INCORPORATED, Gainesville, FL (US)

(72) Inventors: Bala Rathinasabapathi, Gainesville, FL (US); Jingwei FU, Gainesville, FL (US)

(21) Appl. No.: 19/287,459

(22) Filed: Jul. 31, 2025

Related U.S. Application Data
(60) Provisional application No. 63/677,597, filed on Jul. 31, 2024.

(51) Int. Cl. C12N 15/82

(52) U.S. Cl. CPC C12N 15/825

(57) ABSTRACT

Disclosed herein are plants, namely tomato plants, that have increased carotenoid content and that of beta-carotene and improved nutritional value and improved fruit yield. Also disclosed are methods and materials for producing plants with increased carotenoid content, fruit yield and nutritional value.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 15, 2025, is named “10457-601US1.xml” and is 9,841 bytes in size. The sequence listing contained in this .XML file is part of the specification and is hereby incorporated by reference herein in its entirety.

### BACKGROUND

Pepper fruit, a nutrient-dense vegetable for human consumption, contains health-promoting metabolites, including vitamins, alkaloids, and specialized metabolites such as capsanthin, and capsorubin (Wahyuni et al., 2013). Capsanthin and capsorubin are ketocarotenoids, accumulating in the ripe fruit of Capsicum and a few other taxa (Trajković et al., 2021). Capsanthin/capsorubin synthase (CCS) is responsible for the biosynthesis of capsanthin and capsorubin using antheraxanthin and violaxanthin as substrates, respectively (Bouvier et al., 1994; Mialoundama et al., 2010). CCS was first identified from pepper (Bouvier et al., 1994), and validated through enzymatic assays in vitro (Mialoundama et al., 2010). In addition to k-cyclase activity, CCS was reported to have the catalytic activity of lycopene beta-cyclase (LCYB) also, via in vitro assays converting lycopene into β-carotene (Cunningham et al., 1994; Bouvier et al., 1997; Mialoundama et al., 2010). In pepper fruit, the majority of capsanthin and capsorubin are esterified by a xanthophyll acyltransferase (XAT) and stored inside the plastoglobule (Berry et al., 2019). The xanthophyll esterification to fatty acyl groups improves their accumulation, stability, and storage (Ariizumi et al., 2014; Li et al., 2023). However, the relationship between the biosynthesis of abundant ketocarotenoids and the process of xanthophyll esterification in pepper fruit is still unclear. The role of CCS in the carotenoid pathway of ripe pepper fruit remains poorly understood, especially in vivo, although CCS was known to have lycopene cyclase and k-cyclase activities and be associated with xanthophyll esterification process. We hypothesized that transferring CCS from pepper to other related plant species could allow us to test CCS' role in carotenoid biosynthetic flux and accumulation, and the production of specialized ketocarotenoids, and their esters. To date, stable genetic transformation of plants expressing CCS in the endosperm of rice (Ha et al., 2019) and in the flower of Viola cornuta (Trajković et al., 2021) have been reported. However, the alteration of the carotenoid pathway by CCS in a fleshy fruit has remained elusive.

Metabolic engineering is an efficient method to biofortify crops in a targeted direction (Van Der Straeten et al., 2020), such as purple tomato engineered to accumulate anthocyanins (Butelli et al., 2008). Vitamin A is indispensable for human health since vitamin A deficiency (VAD) may result in the development of disorders like xerophthalmia, blindness, and anemia (Blaner, 2020). Globally, an estimated 250-500 million preschool children suffer from VAD (World Health Organization). Hence, carotenoid biofortification of nutrient-dense plant-based foods is an excellent approach to simultaneously combat food insecurity and VAD (Kraemer et al., 2008; Grune et al., 2010; Fitzpatrick et al., 2012; Blaner, 2020).

Multiple cases of carotenoid pathway engineering have been performed to biofortify crops with β-carotene, the major pro-vitamin A (Giuliano, 2017; Zheng et al., 2020), including the first example, golden rice, in which β-carotene accumulated in rice endosperm (Ye & Beyer, 2000). In addition to pro-vitamin A, the alteration of non-pro-vitamin A carotenoids, like lycopene, lutein, and zeaxanthin, through metabolic engineering is also of interest. The pepper ketocarotenoids, capsanthin and capsorubin, are reported to have higher antioxidant ability and potential health promoting activities tested by cell culture and animal model studies (Nishino et al., 2016; Kennedy et al., 2021a). CCS's lycopene cyclase activity has the potential to promote β-carotene biosynthesis. This activity has the potential to promote β-carotene biosynthesis in tissues where lycopene accumulates. Since the utility of overexpressing CCS in a fleshy fruit has not been explored yet we aimed to investigate metabolic changes in the carotenoid pathway, following overexpression of pepper CCS in tomato, as a promising carotenoid biofortification strategy.

### BRIEF DESCRIPTION OF DRAWINGS

FIG. 1. Characterizing the role of capsanthin/capsorubin synthase (CCS) in pepper. (a) The phenotype of ccs-silenced pepper fruit and relative expression level of CCS gene in ccs-silenced pepper fruits compared to mock-treated. Mock treatment refers to the pepper plants infected with VIGS-empty vector without harboring the gene sequence of interest (CaCCS). Data of quantitative RT-qPCR are presented as mean±standard deviation of four biological replicates and three technical replicates. An unpaired t-test was performed (*: P<0.05; **: P<0.01). (b) HPLC chromatograms of mock or ccs-silenced pepper fruit carotenoids through the VIGS system. HPLC analyses were performed without saponification. ‘*’ indicated the derivatives of capsanthin and capsorubin (Kim et al., 2009). (c) Total carotenoids and their compositions in pepper fruit. HPLC analyses were performed without saponification. (d-e) Carotenoid compositions in the mock or ccs-silenced pepper fruits pepper fruit determined after saponification. Data are presented as mean±standard deviation of four biological replicates. Two-way ANOVA was performed followed by Šidik's multiple comparisons test (*: P<0.05; **: P<0.01; ***: P<0.001). (f) The carotenoid pathway in plants. The pre cursor, geranylgeranyl diphosphate, is synthesized from the methylerythritol 4-phosphate (MEP) pathway. Phytoene synthase (PSY) is the first committed step leading to the carotenoid phytoene. Lycopene is synthesized from phytoene after multiple steps, including desaturation and isomerization. Subsequently, lycopene is converted into α-carotene or β-carotene forming two sub-branches, α- and β-branches in the following steps, highlighted in green and yellow color backgrounds, respectively. In the β-branch, the specific steps in red pepper fruit, produce specialized ketocarotenoids, capsanthin and capsorubin, to generate the sub-β-branch. LCYB, lycopene β-cyclase; LCYE, lycopene α-cyclase, CYP97A/C, cytochrome P450-type monooxygenase 97A/C; BCH, β-Carotene hydroxylase; ZEP, zeaxanthin epoxidase; the sub-β-branch indicate the carotenoid flow in pepper fruit.

FIG. 2. Phenotypes and carotenoid profiles of tomato fruit. Overexpression of pepper CCS was performed in ‘Micro-Tom’ background (WT) (a-d). (a) The phenotypes of tomato fruits from ‘Micro-Tom’ WT, ‘Micro-Tom’ transformed with empty vector, WTEV21, and a representative line, WTCCS46, from multiple independent CCS-transformed lines in ‘Micro-Tom’ WT background. (b-d) The corresponding HPLC chromatograms to ‘MicroTom’ WT, WTEV21, and WTCCS46 lines.

FIG. 3. Compositional alterations in the carotenoid profiles of tomato in CCS-transformed tomato. (a) The carotenoid pathway in plants is listed in the black box at the center of this figure. Two branches, α and β-branch, are highlighted in green and yellow backgrounds.

The xanthophylls in the dash-line box were available to be esterified under the catalysis of xanthophyll acyltransferase (XAT). Total carotenoids (b) and lycopene contents (c) in the fruit of control and independent CCS-transgenic lines WT ‘MicroTom’ background. (d) Carotenoid compositions, including lutein and α-carotene, in the α-branch of carotenoid pathway. (e) Carotenoid compositions, including β-carotene, β-cryptoxanthin, zeaxanthin, antheraxanthin, violaxanthin, neoxanthin, capsanthin and capsorubin in the β-branch of carotenoid pathway. (f) A zoom-in view of carotenoids in β-branch from (e) except for β-carotene. (g) Xanthophyll esters in the fruit of control and independent CCS-transgenic lines in both WT ‘MicroTom’ background. Data of b-g are presented as mean±standard deviation of a minimum of four biological replicates based on data from HPLC analyses. One-way ANOVA was performed followed by mean separation test using the Tukey test (*: P<0.05; **: P<0.01; ***: P<0.001).

FIG. 4. Pearson correlation matrix between each paired carotenoid levels analyzed from ripe tomato fruit. The Pearson correlation coefficient of each paired carotenoid composition is shown in each cell computed based on the carotenoid contents from the set of WT, WTEV and WTCCS lines (a) using a R package ‘corrplot’. The Pearson correlation coefficient shown in the cell indicates that it was tested significantly (p-value<0.05), while a tested insignificant correlation would not be shown in the cell. The color intensity and the size of each circle indicated the strength of correlation between each paired carotenoids, in which the more intense the color and larger the circle size are, higher the correlation between each paired carotenoids is. Red indicates positive correlations and blue indicates negative correlations.

FIG. 5. Phenotypic characterization of ripe fruit and carotenoid compositions of parental lines, control- and CCS-derived F1 hybrids. (a) Phenotypes of ripe fruit from parental lines, control- and CCS-derived hybrids. Five tomato inbred lines, ‘FL7907B’, ‘LA2377’, ‘LA2374’, ‘Jaune Flamme’, and ‘LA4044’, were selected as maternal lines. The transgenic line, WTCCS46, generated in this study and ‘MicroTom’ (WT background) were used as paternal lines. (b) Total carotenoids and their compositions in the fruit of parental lines, control- and CCS-derived F1 hybrids. Data are presented as mean±standard deviation of a minimum of four biological replicates based on HPLC analyses. One-way ANOVA was performed followed by mean separation using the Tukey test (*: P<0.05; **: P<0.01; ***: P<0.001). The asterisk color, yellow, pink, and black indicate the mean comparisons of β-carotene, lycopene, and total amounts of carotenoids, respectively.

FIG. 6. Contents of capsanthin (a) and xanthophyll esters (b) in the fruit of control- and CCS-derived hybrids. Data are presented as mean±standard deviation of a minimum of four biological replicates based on HPLC analyses. One-way ANOVA was performed followed by mean separations using the Tukey test (*: P<0.05; **: P<0.01; ***: P<0.001).

FIG. 7. Retinol activity equivalents (RAE) in different vegetables and fruits. RAE values were computed based on the contents of provitamin A, including β-carotene, β-cryptoxanthin, and α-carotene in tomato fruit of engineered lines in this study. The RAE values from different vegetables and fruits, including common sources, were grouped into four, distinguished by dash lines in the figure. The first group depicts data for engineered tomato lines with ‘MicroTom’ WT background. The second group contains the parental lines and their derived F1 hybrids. The parental lines included WT ‘MicroTom’ and ‘WTCCS MicroTom’ (paternal) and five selected inbreds (maternal) ‘FL7907B’, ‘LA2377’, ‘Juane Flamme’, ‘LA2374’, and ‘LA4044’. The third group referred to the RAE values of different vegetables and fruits collected from the USDA database (standard reference legacy).

FIG. 8. Schematic representation of phenotypic and metabolic alteration in CCS-transformed tomato fruit. The carotenoid pathway in WT tomato fruit is shown on the left. The altered carotenoid pathway by overexpression of pepper CCS in tomato fruit is shown on the right. The size of the arrow and each carotenoid represent the relative flux size and metabolite abundance in the pathway. Both pathways are shown in the presence of the xanthophyll esterification process. The accumulation of xanthophyll esters, highlighted in orange box, was positively correlated with the amounts of total carotenoids, β-carotene and other carotenoids, indicating its ability to facilitate carotenoid biosynthesis when CCS was overexpressed. Overexpression of pepper CCS in different tomato varieties (represented here with different color tomatoes), contributed to metabolic alterations of the carotenoid pathway and remarkably enhanced biofortification of provitamin A (β-carotene) for human nutrition. LCYB, lycopene β-cyclase; ZEP, zeaxanthin epoxidase; CCS, capsanthin/capsorubin synthase, highlighted in red. Design components of the figure were obtained from ‘BioRender’ and further assembled in PowerPoint.

FIG. 9. Postharvest rotting of fruit of control (left, LA2377x46) and CCS-expressing tomato (right, LA2377x46CCS) after incubating ripe fruit for 44 days under controlled temperature storage.

FIG. 10. Mean vegetative biomass (a), root biomass (b) and fruit yield (c) of vector control transgenic tomato (WTEV21) and three independent transformants expressing CCS (WTCCS46, WTCCS59 and WTCCS93). Mean values were from 5 replicate plants and the error bars indicate standard deviation. Mean comparisons of CCS-expressing plants against the vector control were done using Dunnett test following one-way ANOVA and results are indicated using asterix (‘*’ significant at P=0.05 and ‘**’ significant at P=0.01).

FIG. 11. Changes to metabolite concentrations in CCS-transformed tomato fruit from three independent lines (WTCCS46, WTCCS59 and WTCCS93) compared to fruit of control line WTEV21. Bars represent mean values of metabolites measured in ripe fruit (n=4), and the error bars represent standard deviation values. Mean comparisons of CCS-expressing plants against the vector control were done using the Dunnett test following one-way ANOVA and results are indicated using asterix (* significant at P=0.05 and ** significant at P=0.01). (a) Reducing sugars, (b) Total amino acids, (c) Total phenolics (mg gallic acid equivalent per 100 grams of dry weight), and (d-e) Total and reduced ascorbic acid.

FIG. 12. ABA and ACC content of ripe tomato fruit from control line WTEV21 compared to CCS-overexpression lines WTCCS46, WTCCS59 and WTCCS93. The mean values for (a) Abscisic acid; (b) ABA-glycosyl ester, (c) 1-Aminocyclopropane-1-carboxylic acid (n=3) with standard deviation bars are shown. The values of WTEV21 line were not significantly different (at P=0.05) from those for fruit of CCS-overexpression lines WTCCS46, WTCCS59 and WTCCS93.

FIG. 13. Volatile apocarotenoid derived from carotenoids in ripe tomato from control and CCS-derived hybrids. Beta-ionone (a) and β-cyclocitral (b) are derived from β-carotene which is highlighted in orange background. Geranial (c), 6-methyl 5-hepten-2-one (d), 6-methyl, 5-hepten-2-ol (e), and neral (f) are derived from lycopene, highlighted in red background. Beta-damascenone (g) and geranyl acetone (h) are derived from neoxanthin and phytoene respectively, highlighted in gray and blue backgrounds respectively. Data are from 100 g fruit for each plant analyzed by individual runs of GC-MS. A paired T-test between the control-derived hybrid group and CCS-derived hybrid group (n=5). P values by paired T-tests were 0.01093 (a), 0.00209 (b), 0.06898 (c), 0.07504 (d), 0.32285 (e), 0.70477 (f), 0.95061 (g), 0.01917 (h). ‘FL7907B’, ‘LA2377’, ‘Flamme’, ‘LA2374’, and ‘LA4044’ represent the maternal lines crossed with WT ‘MicroTom’ (red dots) or ‘WTCCS’ (orange dots).

### DETAILED DESCRIPTION

The present disclosure and embodiments are based on studies investigating the overall role of CCS in the carotenoid pathway of ripe pepper fruit. CCS posed a positive influence on the carotenoid biosynthetic flux, directing it toward the β-branch and xanthophyll acyl esters. Taking advantage of multiple functions of CCS, pepper CCS was over-expressed in ‘Micro-Tom’ WT background. CCS altered the compositions and the flux in the carotenoid pathway of CCS-transformed tomato fruit, resulting in higher accumulation of total carotenoids, β-carotene, and xanthophyll esters. Furthermore, CCS was introduced into five different tomato inbreds through controlled crosses, which resulted in remarkable improvements of β-carotene levels. It was demonstrated that the tomato fruit biofortified with high pro-vitamin A and ketocarotenoids would be an excellent dietary source for human nutrition and to address VAD.

In one embodiment, provided is a method of increasing carotenoid content in a plant (e.g. tomato) compared to a wild type plant. The method involves producing a transgenic plant that includes a recombinant DNA construct that comprises a polynucleotide sequence comprising SEQ ID NO: 1 or a variant thereof like the one shown in SEQ ID NO: 2. The recombinant DNA construct may include a promoter that is functional in a plant cell and operably-linked to the polynucleotide sequence. The plant may be produced by transforming a plant cell or tissue with the recombinant DNA construct, and regenerating or developing the transgenic plant from the plant cell or tissue comprising the recombinant DNA construct. The method may further involve producing a progeny plant comprising the recombinant DNA construct by crossing the transgenic plant with: a) itself, b) a second plant from the same plant line; c) a wild species compatible to cross with tomato or d) a second plant from a different plant line, to produce a seed, growing the seed to produce a progeny plant; and selecting a progeny plant with increased carotenoid content as compared to a control plant.

In a more specific embodiment, the transgenic plant is produced by integration of the recombinant DNA construct into the genome of a plant cell or tissue using a donor template comprising the recombinant DNA construct, and regenerating or developing the transgenic plant from the plant cell or tissue comprising the recombinant DNA construct. Also provided is a transgenic plant produced by the methods described herein.

According to another embodiment, disclosed is recombinant DNA construct that comprises a polynucleotide sequence comprising SEQ ID NO: 2, or a variant thereof. The recombinant DNA construct may further include a promoter functional in a plant cell and operably linked to the polynucleotide sequence. Also disclosed are a vector or plasmid including the recombinant DNA construct. In other embodiments, disclosed is a plant that includes the recombinant DNA construct. In a specific embodiment, the plant may be tomato.

According to other embodiments, provided is commodity product made from the transgenic plants or part thereof, described herein, wherein the plant comprises fruit comprising enhanced carotenoid content as compared to carotenoid content in fruit from a wild-type plant. In a further embodiment, the fruit comprises enhanced levels of ascorbic acid as compared to fruit from a wild-type plant.

In a further embodiment, provided is a transgenic plant that comprises enhanced fruit yield as compared to a wild-type plant and/or a transgenic plant that comprises fruit with enhanced volatile organic compound profiles compared to fruit of a wild-type plant. One embodiment, provided is a transgenic plant that comprises fruit that have better keeping quality and ascorbic acid content.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

The term “Agrobacterium” as used herein refers to a genus of gram-negative bacteria that uses horizontal gene transfer to cause tumors in plants. Agrobacterium tumefaciens is a soil phytopathogen and the most studied species in this genus, which naturally infects plant's wound sites and causes crown gall disease via transfer of a portion of the tumor-inducing (Ti) plasmid, the transfer-DNA (T-DNA), from bacterial cells into host plant cells through a bacterial type IV secretion system (T4SS). (Hwang H H, Yu M, Lai E M. Agrobacterium-mediated plant transformation: biology and applications. Arabidopsis Book. 2017 Oct. 20; 15:e0186).

Ti plasmid is an extrachromosomal molecule of DNA found commonly in the plant pathogen, including Agrobacterium tumefaciens and other species of Agrobacterium such as A. rubi, A. vitis and A. rhizogenes. In the Ti-plasmid, T-DNA is flanked by two repeated sequences of 25-bp length, each, Left and Right Border repeats. These sequences are recognized and nicked by the endonucleolytic proteins VirD1 and VirD2, resulting in a single-stranded form of T-DNA. The VirD2 protein remains covalently linked to the single-stranded T-DNA, and guides it to enter the nucleus. The single-stranded T-DNA is subsequently converted to double-stranded DNA in the nucleus, which may transiently stay in the nucleus to transiently express the genes in the T-DNA or is randomly integrated into the plant genome. (Gelvin S B (2021) Plant DNA Repair and Agrobacterium T-DNA Integration. Int J Mol Sci 22 (16). doi:ARTN 845810.3390/ijms22168458).

As used herein a “control plant” means a plant that does not contain the recombinant DNA of the present disclosure that imparts an enhanced trait or altered phenotype. A control plant is used to identify and select a transgenic plant that has an enhanced trait or altered phenotype. A suitable control plant can be a non-transgenic plant of the parental line used to generate a transgenic plant, for example, a wild type plant devoid of a recombinant DNA. A suitable control plant can also be a transgenic plant that contains recombinant DNA that imparts other traits, for example, a transgenic plant having enhanced herbicide tolerance. A suitable control plant can in some cases be a progeny of a hemizygous transgenic plant line that does not contain the recombinant DNA, known as a negative segregant, or a negative isogenic line.

As used herein an “enhanced trait” means a characteristic of a transgenic plant as a result of stable integration and expression of a recombinant DNA in the transgenic plant. In a specific example, the trait is increased carotenoid content, or more specifically, pro-vitamin A content as compared to a control plant.

The term “genome editing” refers to modifying a genome with techniques that employ targeted mutagenesis to activate DNA repair pathways. These techniques include, but are not limited to, those that utilize endonucleases to generate single-strand and double-strand DNA breaks that activate DNA repair pathways. Genome editing techniques may also comprise systems that enable targeted editing at any genomic locus. These targeting systems include, but are not limited to, polypeptides, such as, Transcription Activator-Like Effectors nucleases (TALENs) and zinc finger nucleases (ZFNs), or nucleic acids, such as, Clustered Regularly Interspaced Short Palindromic Repeats/Cas (CRISPR/CAS) single guide RNAs or NgAgo (Argonaute) single strand DNAs. As used herein, “genome editing” and “genome-engineering” are interchangeable.

The term “genetic modification” refers to a DNA sequence difference, epigenetic difference, or combination thereof between two genomes of the same species in which one genome is identified as the modified genome and the other is identified as the unmodified genome and the DNA sequence or epigenetic difference is the result of applying genome modifying techniques to the unmodified genome to yield the modified genome. A genetic modification, as used herein, encompasses any insertion, deletion, or substitution of a nucleotide sequence of any size and nucleotide content, any epigenetic modification to any number of nucleotides, or a combination thereof. A genetic modification, as used herein, may also encompass introduction of one or more exogenous coding nucleic acids that do not integrate into the unmodified genome, yet are capable of autonomous replication. In certain embodiments, a modification to an endogenous gene or regulatory element thereof may be a deletion, a substitution, or an insertion that reduces expression of the endogenous gene or the polypeptide for which it encodes. In specific embodiments, the modification may be an indel, wherein the indel may cause a frameshift mutation, a missense mutation, a nonsense mutation, a neutral mutation, or a silent mutation. In specific embodiments, a modification to a regulatory element of an endogenous gene may alter or eliminate a function of the regulatory element. In further contemplated embodiments, the modification may comprise a nucleic acid sequence that provides exogenous control of endogenous gene, mRNA, or polypeptide expression levels. In specific embodiments, the modification may also disrupt a post-translational process of a polypeptide encoded by an endogenous gene. Post-translational processes in certain embodiments may be post-translational protein modification, protein sorting, or proteasomal degradation.

The term “operably linked” refers to a first nucleic acid sequence connected with a second nucleic acid sequence when the first nucleic acid sequence is placed in a functional relationship with the second nucleic acid sequence. For example, a promoter sequence is “operably linked” to a DNA if the promoter provides for transcription or expression of the DNA. Generally, operably linked DNA sequences are contiguous.

The term “plant” includes whole plants, plant organs (e.g., leaves, stems, roots, flowers fruit, etc.), seeds and plant cells and progeny of same. The class of plants which can be used in the methods of the invention is generally as broad as the class of higher plants amenable to transformation techniques, including both monocotyledonous and dicotyledonous plants. In a specific embodiment, the plant is tomato, Solanum lycopersicum.

As used herein, the term “promoter” refers generally to a DNA molecule that is involved in recognition and binding of RNA polymerase II and other proteins (trans-acting transcription factors) to initiate transcription. A promoter can be initially isolated from the 5′ untranslated region (5′ UTR) of a genomic copy of a gene. Alternately, promoters can be synthetically produced or manipulated or engineered DNA molecules. Promoters can also be chimeric, that is a promoter produced through the fusion of two or more heterologous DNA molecules. Plant promoters include promoter DNA obtained from plants, plant viruses, fungi and bacteria such as Agrobacterium and Bradyrhizobium bacteria. A promoter may also be heterologous. As used herein, a promoter or other regulatory sequence operably linked to a transcribable DNA sequence, such as a coding sequence, is considered “heterologous” if in nature the promoter or regulatory sequence is not operably linked to the transcribable DNA sequence and/or is not present in the plant host cell to be transformed with the promoter or regulatory sequence. Two or more promoter or regulatory sequences may also be heterologous with respect to each other.

Promoters which initiate transcription in all or most tissues of the plant are referred to as “constitutive” promoters. Promoters which initiate transcription during certain periods or stages of development are referred to as “developmental” promoters. Promoters whose expression is enhanced in certain tissues of the plant relative to other plant tissues are referred to as “tissue enhanced” or “tissue preferred” promoters. Promoters which express within a specific tissue of the plant, with little or no expression in other plant tissues are referred to as “tissue specific” promoters. A promoter that expresses in a certain cell type of the plant, for example a microspore mother cell, is referred to as a “cell type specific” promoter. An “inducible” promoter is a promoter in which transcription is initiated in response to an environmental stimulus such as cold, drought or light; or other stimuli such as wounding or chemical application. Many physiological and biochemical processes in plants exhibit endogenous rhythms with a period of about 24 hours. A “diurnal promoter” is a promoter which exhibits altered expression profiles under the control of a circadian oscillator. Diurnal regulation is subject to environmental inputs such as light and temperature and coordination by the circadian clock. Many examples of plant expressible promoters are known in the art.

A “recombinant DNA construct” as used in the present disclosure comprises at least one expression cassette having a promoter operable in plant cells and a polynucleotide of the present disclosure. DNA constructs can be used as a means of delivering recombinant DNA constructs to a plant cell in order to effect stable integration of the recombinant molecule into the plant cell genome. In one embodiment, the polynucleotide can encode a protein or variant of a protein or fragment of a protein that is functionally defined to maintain activity in transgenic host cells including plant cells, plant parts, explants and whole plants. Recombinant DNA constructs are assembled using methods known to persons of ordinary skill in the art and typically comprise a promoter operably linked to DNA, the expression of which provides the enhanced agronomic trait.

Other construct components can include additional regulatory elements, such as 5′ leaders and introns for enhancing transcription, 3′ untranslated regions (such as polyadenylation signals and sites), and DNA for transit or targeting or signal peptides.

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

The term “transformation” or “transform” refers to genetic transformation that is a process that involves the introduction and expression of foreign genes in a host organism. This expression can result from the extrachromosomal, or episomal presence of genes in nuclei that may persist if the introduced DNA has a mechanism for replication. Bacterial transformation is a process of horizontal gene transfer by which some bacteria take up foreign genetic material (naked DNA) from the environment. For example, in some embodiments, Agrobacterium is transformed with plasmids comprising T-DNA fragment, using heat and thaw methods. Plant genetic transformation (PGT) is a process where DNA is introduced into plant cells, tissues, or organs using molecular and cellular biology methods. PGT comprises steps of delivery of the DNA into a single cell and regeneration into entire fertile plants. In some embodiments, explants are genetically ‘transformed’ to have a specific gene modified and express altered phenotypes using Agrobacterium ‘transformed’ with plasmids encoding CRISPR/Cas gene editing system. Transformation protocols as well as protocols for introducing polypeptides or polynucleotide sequences into plants may vary depending on the type of plant or plant cell, i.e., monocot or dicot, targeted for transformation.

Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec transformation (WO 00/28058). D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The term “transgenic plant” as used herein refers to the plant that have been genetically modified by the insertion of a foreign genetic material (gene(s), DNA sequence(s), etc.) into the chromosome of the cell, while a “transformed plant” is the one whose genome has been genetically modified but not necessarily through insertion of a foreign genetic material. A transgenic plant may have an altered genome containing a DNA sequence or gene from a different species, which expresses a protein that is not native to the plant. The protein encoded by the gene will confer a particular trait or characteristic to that plant.

As used herein, the term “variants” refers to nucleic acid or polypeptide sequences having substantial similarity with a sequence disclosed herein. A variant may comprise a deletion and/or addition of one or more nucleotides or peptides at one or more internal sites within the native polynucleotide or polypeptide and/or a substitution of one or more nucleotides or peptides at one or more sites in the native polynucleotide or polypeptide. Variant nucleotide sequences also include synthetically derived nucleotide sequences, such as those generated, for example, by using site-directed mutagenesis. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to a reference nucleotide sequence as determined by sequence alignment programs described elsewhere herein using default parameters. In preferred embodiments, a variant will have at least 90%, 91%, 92%, 93%, 94%, to 95%, 96%, 97%, 98%, 99% or more sequence identity to a reference sequence. Biologically active variants of a nucleotide sequence disclosed herein are also encompassed. Biological activity may be measured by using techniques such as Northern blot analysis, reporter activity measurements taken from transcriptional fusions, and the like. See, for example, Sambrook, et al., (1989) Molecular Cloning: A Laboratory Manual (2d ed., Cold Spring Harbor Laboratory Press, Cold Spring Harbor, N.Y.), hereinafter “Sambrook”, herein incorporated by reference in its entirety. Alternatively, levels of a marker gene such as green fluorescent protein (GFP) or yellow fluorescent protein (YFP) or the like produced under the control of a promoter operably linked to a nucleotide fragment or variant can be measured. See, for example, Matz et al. (1999) Nature Biotechnology 17:969-973; U.S. Pat. No. 6,072,050, herein incorporated by reference in its entirety; Nagai, et al., (2002) Nature Biotechnology 20(1):87-90.

The term “vector” or “vector system” as used herein means a transport means which can introduce a recombinant construct (comprising nucleic acids or even polypeptides as well as further sequences such as regulatory sequences or localization sequences) directly or indirectly into a desired target cell or target plant structure, into the desired cellular compartment.

The term “vector system” as used here denotes a system which consists of at least one or more vector(s) or contains it(them). Thus, a vector system may comprise a vector which contains/codes for two different recombinant constructs comprising nucleic acid and/or amino acid sequences. Furthermore, a vector system can also contain several vectors which in their turn contain/code for at least one nucleic acid or amino acid sequence in accordance with the present disclosure.

### Exemplary Sequences

Polynucleotide sequence coding for Capsanthin/capsorubin
synthase.
NCBI accession number: PP418871. The protein coding
sequence is indicated by an underline.
SEQ ID NO: 1
ATGGAAACCCTTCTAAAGCCTTTTCCATCTCCTTTACTTTCCATTCCTACTCCTAACAT
GTATAGTTTCAAACACAACTCCACTTTTCCAAATCCAACCAAACAAAAAGATTCAAG
AAAGTTCCATTATAGAAACAAAAGCAGTACACATTTTTGTAGCTTTCTTGATTTAGCAC
CCACATCAAAGCCAGAGTCTTTAGATGTTAACATCTCATGGGTTGATACTGATCTGGA
CGGGGCTGAATTCGACGTGATCATCATTGGAACTGGCCCTGCCGGGCTTCGGCTAGCT
GAACAAGTTTCTAAATATGGTATTAAGGTATGTTGCGTTGACCCTTCACCACTTTCCAT
GTGGCCAAATAATTATGGTGTTTGGGTTGATGAGTTTGAAAAGTTGGGATTAGAAGAT
TGTCTAGATCATAAGTGGCCTGTGAGTTGTGTTCATATAAGTGATCACAAGACTAAGT
ATTTGGACAGACCATATGGTAGAGTAAGTAGAAAGAAGTTGAAGTTGAAATTGTTGA
ATAGTTGTGTTGAAAATAGAGTGAAGTTTTATAAAGCCAAGGTTTTGAAAGTGAAGC
ATGAAGAATTTGAGTCTTCGATTGTTTGTGATGATGGTAGGAAGATAAGCGGTAGCTT
GATTGTTGATGCAAGTGGCTATGCTAGTGATTTTATAGAGTATGACAAGCCAAGAAAC
CATGGTTATCAAGTTGCTCATGGGATTTTAGCAGAAGTTGATAATCATCCATTTGATTT
GGATAAAATGATGCTTATGGATTGGAGGGATTCTCATTTAGGTAATGAGCCATATCTGA
GGGTGAAGAATACTAAAGAACCAACATTCTTGTATGCAATGCCATTTGATAGGAATTT
GGTATTCTTGGAAGAGACTTCTTTAGTGAGTCGGCCTATGTTATCGTATATGGAAGTGA
AAAGAAGGATGGTAGCAAGATTAAGACATTTGGGGATCAAAGTGAGAAGTGTCCTTG
AGGAAGAGAAGTGTGTGATCACTATGGGAGGACCACTTCCGCGGATTCCTCAAAATG
TTATGGCTATTGGTGGGACTTCAGGGATAGTTCATCCATCGTCTGGGTACATGGTGGCT
CGTAGCATGGCATTGGCACCAGTACTGGCTGAGGCCATCGTCGAAAGCCTTGGCTCA
ACAAGAATGATAAGAGGGTCTCAACTTTACCATAGAGTTTGGAATGGTTTGTGGCCTT
CGGATAGAAGACGTGTTAGAGAATGTTATTGTTTCGGAATGGAGACTTTGTTGAAGCT
TGATTTGGAAGGTACTAGGAGATTGTTTGATGCTTTCTTTGATGTTGATCCCAAGTACT
GGCACGGGTTCCTTTCTTCAAGATTGTCTGTCAAAGAACTTGCTGTACTCAGTTTGTA
CCTTTTTGGACATGCCTCTAATTTGGCTAGGTTGGATATTGTTACAAAGTGCACTGTCC
CCTTGGTTAAACTGCTGGGCAATCTAGCAATAGAGAGCCTTTGAATTAATATGATAGTT
TTGAAGCACTGTTTTCATTTTAATTTCTTAGGTTATTTTCATCTTTTCTCAATGCAAAAG
TGAAACAAAAGCTATACACATTGTCATCGTTGTTCAAACTCAGACAAGTTTGCCTAGC
TCTATGTATTTATCCTTAACATATGTATTCATCAAATTCGAAATATACAATGCATTGGACA
AAAGTATAGAGCCACAATCCGATACCAAGTCTGTATTTGGAAGCACTGTCTAATTGTT
ATGGTTACCAAACACTTTGAATTGGCTGGATAATAACAAACAGGAAATTTATGTTTTTA
ATCATTAACAGCAAATTGGGAAAGCAAGA.
Polynucleotide sequence coding for a fusion protein of
Capsanthin/capsorubin synthase (underlined) and Green
Fluorescent Protein (GFP, shown in BOLD text).
SEQ ID NO: 2
ATGGAAACCCTTCTAAAGCCTTTTCCATCTCCTTTACTTTCCATTCCTACTCCTAACAT
GTATAGTTTCAAACACAACTCCACTTTTCCAAATCCAACCAAACAAAAAGATTCAAG
AAAGTTCCATTATAGAAACAAAAGCAGTACACATTTTTGTAGCTTTCTTGATTTAGCAC
CCACATCAAAGCCAGAGTCTTTAGATGTTAACATCTCATGGGTTGATACTGATCTGGA
CGGGGCTGAATTCGACGTGATCATCATTGGAACTGGCCCTGCCGGGCTTCGGCTAGCT
GAACAAGTTTCTAAATATGGTATTAAGGTATGTTGCGTTGACCCTTCACCACTTTCCAT
GTGGCCAAATAATTATGGTGTTTGGGTTGATGAGTTTGAAAAGTTGGGATTAGAAGAT
TGTCTAGATCATAAGTGGCCTGTGAGTTGTGTTCATATAAGTGATCACAAGACTAAGT
ATTTGGACAGACCATATGGTAGAGTAAGTAGAAAGAAGTTGAAGTTGAAATTGTTGA
ATAGTTGTGTTGAAAATAGAGTGAAGTTTTATAAAGCCAAGGTTTTGAAAGTGAAGC
ATGAAGAATTTGAGTCTTCGATTGTTTGTGATGATGGTAGGAAGATAAGCGGTAGCTT
GATTGTTGATGCAAGTGGCTATGCTAGTGATTTTATAGAGTATGACAAGCCAAGAAAC
CATGGTTATCAAGTTGCTCATGGGATTTTAGCAGAAGTTGATAATCATCCATTTGATTT
GGATAAAATGATGCTTATGGATTGGAGGGATTCTCATTTAGGTAATGAGCCATATCTGA
GGGTGAAGAATACTAAAGAACCAACATTCTTGTATGCAATGCCATTTGATAGGAATTT
GGTATTCTTGGAAGAGACTTCTTTAGTGAGTCGGCCTATGTTATCGTATATGGAAGTGA
AAAGAAGGATGGTAGCAAGATTAAGACATTTGGGGATCAAAGTGAGAAGTGTCCTTG
AGGAAGAGAAGTGTGTGATCACTATGGGAGGACCACTTCCGCGGATTCCTCAAAATG
TTATGGCTATTGGTGGGACTTCAGGGATAGTTCATCCATCGTCTGGGTACATGGTGGCT
CGTAGCATGGCATTGGCACCAGTACTGGCTGAGGCCATCGTCGAAAGCCTTGGCTCA
ACAAGAATGATAAGAGGGTCTCAACTTTACCATAGAGTTTGGAATGGTTTGTGGCCTT
CGGATAGAAGACGTGTTAGAGAATGTTATTGTTTCGGAATGGAGACTTTGTTGAAGCT
TGATTTGGAAGGTACTAGGAGATTGTTTGATGCTTTCTTTGATGTTGATCCCAAGTACT
GGCACGGGTTCCTTTCTTCAAGATTGTCTGTCAAAGAACTTGCTGTACTCAGTTTGTA
CCTTTTTGGACATGCCTCTAATTTGGCTAGGTTGGATATTGTTACAAAGTGCACTGTCC
CCTTGGTTAAACTGCTGGGCAATCTAGCAATAGAGAGCCTTAAGGGTGGGCGCGCCG
ACCCAGCTTTCTTGTACAAAGTGGTGATATCAATGGTGAGCAAGGGCGAGGAGCTG
TTCACCGGGGTGGTGCCCATCCTGGTCGAGCTGGACGGCGACGTAAACGGCCA
CAAGTTCAGCGTGTCCGGCGAGGGCGAGGGCGATGCCACCTACGGCAAGCTGA
CCCTGAAGTTCATCTGCACCACCGGCAAGCTGCCCGTGCCCTGGCCCACCCTC
GTGACCACCCTGACCTACGGCGTGCAGTGCTTCAGCCGCTACCCCGACCACAT
GAAGCAGCACGACTTCTTCAAGTCCGCCATGCCCGAAGGCTACGTCCAGGAGC
GCACCATCTTCTTCAAGGACGACGGCAACTACAAGACCCGCGCCGAGGTGAAG
TTCGAGGGCGACACCCTGGTGAACCGCATCGAGCTGAAGGGCATCGACTTCAA
GGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGA
TCCGCCACAACATCGAGGACGGCAGCGTGCAGCTCGCCGACCACTACCAGCAG
AACACCCCCATCGGCGACGGCCCCGTGCTGCTGCCCGACAACCACTACCTGAG
CACCCAGTCCGCCCTGAGCAAAGACCCCAACGAGAAGCGCGATCACATGGTCC
TGCTGGAGTTCGTGACCGCCGCCGGGATCACTCTCGGCATGGACGAGCTGTAC
AAGTAA.
Deduced amino acid sequence of Capsanthin/Capsorubin
synthase. NCBI accession number: WWQ39373.1
SEQ ID NO: 3
METLLKPFPSPLLSIPTPNMYSFKHNSTFPNPTKQKDSRKFHYRNKSSTHFCSFLDLAPTS
KPESLDVNISWVDTDLDGAEFDVIIIGTGPAGLRLAEQVSKYGIKVCCVDPSPLSMWPNN
YGVWVDEFEKLGLEDCLDHKWPVSCVHISDHKTKYLDRPYGRVSRKKLKLKLLNSCVE
NRVKFYKAKVLKVKHEEFESSIVCDDGRKISGSLIVDASGYASDFIEYDKPRNHGYQVAH
GILAEVDNHPFDLDKMMLMDWRDSHLGNEPYLRVKNTKEPTFLYAMPFDRNLVFLEET
SLVSRPMLSYMEVKRRMVARLRHLGIKVRSVLEEEKCVITMGGPLPRIPQNVMAIGGTS
GIVHPSSGYMVARSMALAPVLAEAIVESLGSTRMIRGSQLYHRVWNGLWPSDRRRVREC
YCFGMETLLKLDLEGTRRLFDAFFDVDPKYWHGFLSSRLSVKELAVLSLYLFGHASNLA
RLDIVTKCTVPLVKLLGNLAIESL.
Deduced amino acid sequence of the fusion protein that codes
for Capsanthin/Capsorubin synthase - Green Fluorescent
Protein (GFP) fusion. eGFP part is indicated by bold letters.
SEQ ID NO: 4
METLLKPFPSPLLSIPTPNMYSFKHNSTFPNPTKQKDSRKFHYRNKSSTHFCSFLDLAPTS
KPESLDVNISWVDTDLDGAEFDVIIIGTGPAGLRLAEQVSKYGIKVCCVDPSPLSMWPNN
YGVWVDEFEKLGLEDCLDHKWPVSCVHISDHKTKYLDRPYGRVSRKKLKLKLLNSCVE
NRVKFYKAKVLKVKHEEFESSIVCDDGRKISGSLIVDASGYASDFIEYDKPRNHGYQVAH
GILAEVDNHPFDLDKMMLMDWRDSHLGNEPYLRVKNTKEPTFLYAMPFDRNLVFLEET
SLVSRPMLSYMEVKRRMVARLRHLGIKVRSVLEEEKCVITMGGPLPRIPQNVMAIGGTS
GIVHPSSGYMVARSMALAPVLAEAIVESLGSTRMIRGSQLYHRVWNGLWPSDRRRVREC
YCFGMETLLKLDLEGTRRLFDAFFDVDPKYWHGFLSSRLSVKELAVLSLYLFGHASNLA
RLDIVTKCTVPLVKLLGNLAIESLKGGRADPAFLYKVVISMVSKGEELFTGVVPILVELD
GDVNGHKFSVSGEGEGDATYGKLTLKFICTTGKLPVPWPTLVTTLTYGVQCFSRYP
DHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQN
TPIGDGPVLLPDNHYLSTQSALSKDPNEKRDHMVLLEFVTAAGITLGMDELYK*.

### Summary of Experimental Results

Biofortification of tomato fruit by a simultaneous accumulation of β-carotene, a pro-vitamin A, and pepper-specialized ketocarotenoids, capsanthin, and capsorubin, leads to favorable modifications of the carotenoid pathway, and nutritionally enhanced fruit for combatting vitamin A deficiency.
Role of capsanthin/capsorubin synthase (CCS) in pepper was investigated via virus-induced gene silencing. Pepper CCS was overexpressed in different tomato varieties, including ‘Micro-Tom’ WT and five selected inbreds to characterize the alteration in fruit carotenoid profiles.
In pepper, silencing of CCS reduced the biosynthetic flux toward the β-branch and showed significant changes in carotenoid compositions. In CCS-transformed tomato fruit (WT and selected varieties), besides the biosynthesis of capsanthin and capsorubin, total carotenoids, β-carotene, and xanthophyll esters remarkably increased compared to the controls, while the mutant showed weaker increments.
In pepper and CCS-transformed tomato, CCS posed a positive influence on the biosynthetic flux toward the β-branch and the storage pool of xanthophyll esters due to its functions in lycopene cyclization and ketocarotenoid biosynthesis. It indicated that xanthophyll esters might facilitate carotenoid biosynthesis. Consumption of 37-131-gram of CCS-derived hybrid fruit meets the pro-vitamin A recommended dietary allowance, indicating greatly improved nutritional value.

### EXAMPLES

### Example 1: Materials and Methods for Examples 2-7

### Plant Materials and Growth Conditions

Seeds of wild-type (WT) Solanum lycopersicum L. cv Micro-Tom. Seeds of LA2374 (‘Caro Red’), LA2377 (‘Purple Calabash’), and LA4044 (Solanum pennellii, IL3-2, (Alseekh et al., 2013) were from the Tomato Genetics Resource Center (University of California, Davis, CA, U.S.A.). Seeds of FL7907B were from Dr. S. Hutton (GCREC, University of Florida, Wimauma, FL, U.S.A.). Seeds of ‘Jaune Flamme’ were from Tomato Grower's Supply Company (Fort Myers, FL, U.S.A.). Seedlings were grown in a potting medium (Jolly Gardener, C25) supplied with controlled-release fertilizer (Florikan, N: K:P=18:6:8). Growing conditions were as follows: 16-h-day/8-h-night photoperiod, 25° C./22° C. day/night temperature, 50% relative humidity, and ˜150 μmol m⁻² s⁻¹ light intensity supplemented by fluorescent lights.

cDNA Cloning

Total RNA was isolated from ripe pepper pericarp of RJ107(6)A3 (Maquilan et al., 2020) using RNeasy plant mini kit (Qiagen, Germany). First-strand cDNA was synthesized using SuperScript®III First-Strand Synthesis System (Invitrogen, U.S.A.). Capsanthin/capsorubin synthase (CCS) protein coding sequence (CDS) minus stop codon were amplified from first strand cDNA using specific primers listed in Table 1. The gel-purified amplicons were cloned into an entry vector pENTR™/D-TOPO® (Invitrogen, U.S.A.). CaCCS cDNA from the entry vector were subcloned into plant expression vector pK7FWG2.0 by Gateway LR recombination reaction (Invitrogen, U.S.A.) to generate pK7FWG2.0-CaCCS-eGFP. Following all cloning operations, sequence verification was done by Sanger sequencing.

TABLE 1

| Name of | | |
|---|---|---|
| primer | Forward sequence (5′ to 3′) | Reverse sequence (5′ to 3′) |
| CaCCS | CACCAGTGATTCCCCTAGTTCGGT | AAGGCTCTCTATTGCTAGATTGC |
| full CDS | ATT | |
| CaCCS | AGGCCTCCATGGCCTTTTCCATCT | CCGGGCCTCGAGCTGTCCAAATACTT |
| VIGS | CCTTTACTT | AGTCTTGTGAT |
| CaCCS | TACTGGCACGGGTTCCTTTC | TGCTAGATTGCCCAGCAGTT |
| qPCR | | |
| CaUbCE | GAGCTCAAGGATCTGCAAAAGGA | GTTCTAAAAGCAACCTTTGGAGGC |
| qPCR | | |

### Virus-Induced Ccs-Silencing in Pepper Fruit

The CCS knock-down pepper lines were generated by the VIGS modified from (Liu et al., 2002). The target region for silencing was designed by the SGN VIGS Tool (https://vigs.solgenomics.net). A 460 bp fragment of CCS was amplified from pepper fruit cDNA using specific primers listed in Table 1. After subcloning into pTRV2-MCS, which was obtained along with pTRV1 from the Arabidopsis Biological Resource Center, pTRV2-CCS was generated. This plasmid was transformed into Agrobacterium tumefaciens strain GV3101. The agroinfiltration of pepper plants was performed as previously described (Kim et al., 2017). Agrobacterium strains GV3101 harboring pTRV2-CCS and pTRV1 were mixed 1:1 and infiltrated onto open cotyledons using 1-mL syringe without needles. The Agrobacterium strain GV3101 containing pTRV2-MCS was used as a control. Infiltrated plants were kept under dark and high humidity conditions overnight, followed by normal conditions.

### Quantitative Real-Time PCR

RNA extraction and single-stranded cDNA were prepared as described in ‘cDNA Cloning’. The expression level of CCS gene was estimated by quantitative RT-PCR using green fast qPCR blue mix (AzuraView™, MA, U.S.A.) with gene-specific primers (Table 1). The relative expression levels of targeted genes were quantified using the equation of 2^{\hat{ }-ΔCT} normalized to the gene encoding ubiquitin-conjugating enzyme E2 (Cheng et al., 2017). Each qPCR reaction was run in four biological and three technical replicates.

### Plant Transformation

Agrobacterium-mediated transformation of tomato was performed using the method of Sun et al., (2006) with modifications. Agrobacterium GV3101 harboring pK7FWG2.0-CaCCS-eGFP were grown in YEP to prepare Agrobacterium inoculum. Surface-sterilized seeds of WT ‘Micro-Tom’ were germinated on half-strength MS medium (Murashige & Skoog, 1962). Cotyledon explants from 10-day old seedlings were placed on a preculture medium for 48 h, blotted dry and placed on a co-cultivation medium at 25° C. in the dark for 48 h. Subsequently, the explants were transferred to the callus induction medium for two weeks and then to shoot induction medium until shoots formed. Elongated shoots were excised and transferred to the rooting medium. Rooted shoots were transplanted to soil. Media components are listed in Table 2. Genomic PCR with T-DNA-specific primers were performed for identifying the putative transformants. The T₀ transgenic lines were grown to collect seeds, and their T₂ generations were used for further analyses.

TABLE 2
Medium for plant transformation. All media were adjusted
to pH 5.7 prior to autoclaving. Plant growth regulators,
antibiotic stocks, acetosyringone, and 2-mercaptoethanol,
were filter-sterilized and added after autoclaving.

| | Components including antibiotics, plant growth regulators |
|---|---|
| Medium | and agar |
| YEP | YEP medium containing 50 mg L⁻¹ spectinomycin, 10 mg |
| medium | L⁻¹ rifampicin, and 20 mg L⁻¹ gentamycin |
| Preculture | MS medium supplemented with 3% (w/v) sucrose, 2 mg L⁻¹ |
| medium | zeatin and 0.8% (w/v) agar |
| Co- | MS medium supplemented with 3% (w/v) sucrose, 2 mg |
| cultivation | L⁻¹ zeatin, 100 μM acetosyringone, 10 μM |
| medium | 2-mercaptoethanol, and 0.8% (w/v) agar |
| Callus | MS medium supplementing with 3% (w/v) sucrose, 2 mg |
| induction | L⁻¹ zeatin, 100 mg L⁻¹ kanamycin, 100 mg |
| medium | L⁻¹ timentin, 300 mg L⁻¹ cefotaxime, and |
| | 0.8% (wt./v) agar |
| Shoot | MS medium supplemented with 3% (w/v) sucrose, 1 mg L⁻¹ |
| induction | zeatin, 100 mg L⁻¹ kanamycin, 100 mg L⁻¹ timentin, |
| medium | 300 mg L⁻¹ cefotaxime, and 0.8% (wt./v) agar |
| Rooting | 0.5 × MS medium supplemented with 1.5% (w/v) sucrose, |
| medium | 1 mg L⁻¹ IAA, 50 mg L⁻¹ kanamycin, 100 mg |
| | L⁻¹ timentin, 300 mg L⁻¹ cefotaxime, and |
| | 0.6% (w/v) agar |

### Control- and CCS-Derived Tomato Lines

Five tomato inbreds, ‘FL7907B’, ‘LA2377’, ‘LA2374’, ‘Jaune Flamme’, and ‘LA4044’, were selected as maternal lines. The transgenic line, WTCCS46, generated in this study and ‘Micro-Tom’ (WT background) were used as paternal lines. Pollen from the WTCCS46 was transferred onto the stigma of emasculated flowers of the selected lines, to generate CCS-derived lines. Similar crosses were made between WT ‘Micro-Tom’ and the selected lines to generate control-derived lines. Seeds of ripe fruit resulting from crosses were collected and germinated to generate the F1 plants. Ten plants from each cross were grown as biological replicates. All F1 plants harboring the transgene were validated by genomic PCR with T-DNA-specific primers. The F1 plants were grown in a temperature-controlled greenhouse in 5-gallon containers filled with potting medium and fertilized with the Tomato Plant Food every week (MiracleGro, Marysville, OH, U.S.A.). The ripe fruit from all lines were collected for analyses.

### Carotenoid Analysis

Carotenoids were extracted from pepper or tomato pericarp following the method of Wahyuni (2011). The pooled organic solvent layers from extracts were combined, dried under a nitrogen stream, and were used either for direct quantification (unsaponified) or for saponification based on the method of Minguez-Mosquera & Hornero-Ménndez (1993). Quantification of carotenoids was performed by HPLC-UV/Vis following the method adapted from Delgado-Pelayo & Hornero-Méndez (2012). The chromatographic system comprised an Agilent 1100 chromatograph connected to an Agilent G1314A UV/Vis detector operated by ChemStation software. The chromatographic conditions are listed in Table 3. Lycopene and β-carotene standards were from Sigma-Aldrich (MO, U.S.A.). Zeaxanthin, lutein, and zeaxanthin dipalmitate were from Cayman. Capsanthin and capsorubin were purified from pepper fruit by column chromatography using the methods of Cvetković & Markovic (2008). Each carotenoid concentration was calculated using the standard curves. The xanthophyll ester fractions were quantified using the calibration curve of zeaxanthin dipalmitate since standards for most other xanthophyll esters were not commercially accessible. Concentration was calculated as μg per gram of fresh weight (μg g⁻¹ f.wt.).

TABLE 3
Chromatographic Method

| Condition | Setting or Specification |
|---|---|
| Main | Thermo Scientific Acclaim RSLC 120 C18 column (250 |
| column | mm × 2.1 mm i.d., 2.2 μm) |
| Guard | A similar material of main column (20 mm × 4.6 mm) |
| column | |
| Temperature | 35° C. |
| Injection | 1-4 μL |
| Flow rate | 0.1 ml/min |
| Detector | The carotenoids were detected at a wavelength of 450 nm, |
| | and the spectra were acquired and processed using the |
| | ChemStation software. |
| Mobile | The mobile phase consisted of eluent A (water) and eluent |
| phase | B (acetone) using a gradient program as follows: 75% B |
| | was increased linearly to 90% B in 20 min, subsequently |
| | holding for 15 min, then raised to 95% B in 10 min, and |
| | maintained for 20 min constantly; finally, eluent B |
| | reached 100% in 15 min and maintained for another |
| | 20 min. Initial conditions were reached in another |
| | 60 min. |

### Example 2: Characterization of Capsanthin/Capsorubin Synthase (CCS) in Pepper Carotenoid Pathway

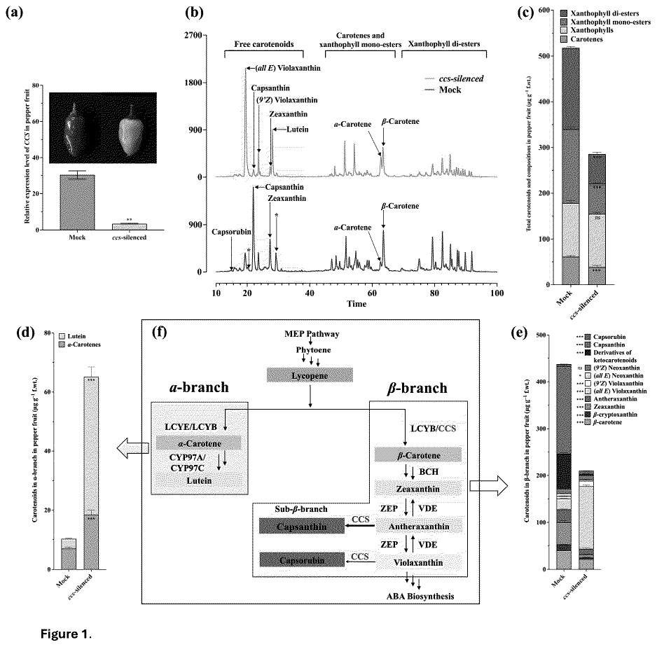

A Pearson correlation analysis on the relationship between the contents of paired carotenoids from 32 pepper varieties adopted from a previous study (Wahyuni et al., 2011) showed that capsanthin and capsorubin contents were positively correlated with β-carotene contents (r=0.88), suggesting a role for CCS in accumulation of capsanthin, capsorubin, and f-carotene in pepper fruit. To confirm CCS' function in the carotenoid pathway, CCS expression in pepper fruit was silenced through virus-induced gene silencing (VIGS). The relative expression of the CCS transcript dropped >10-fold in ccs-silenced fruit compared to the mock, paralleling with the yellow color of ripe fruit as opposed to the red of the mock control (FIG. 1a). The carotenoid profile of ccs-silenced fruit showed a distinguishable difference from that of mock-treated fruit (FIG. 1b). Compared to mock-treated fruit, total carotenoids were significantly reduced in the ccs-silenced fruit, among which xanthophyll esters and carotenes decreased by 61.5% and 37.4% respectively (FIG. 1c, f). Specifically, lutein and α-carotene contents in the α-branch of the carotenoid pathway were elevated by 13.2 and 1.6-fold respectively when CCS expression was silenced (FIG. 1d, f). Oppositely, capsanthin and capsorubin levels in the β-branch were reduced by 96% and 99% respectively in ccs-silenced fruit (FIG. 1f). The silencing of the CCS gene in pepper fruit reduced not only capsanthin and capsorubin, but also β-carotene levels by 46% in pepper fruit (FIG. 1e, f), consistent with the results of correlation analysis. In the β-branch, the amounts of other carotenoids, except for (all E) violaxanthin and (9′Z) violaxanthin, were reduced significantly as well in ccs-silenced fruit (FIG. 1e, f).

### Example 3: Phenotypic Alteration of CCS-Overexpression Tomato Lines

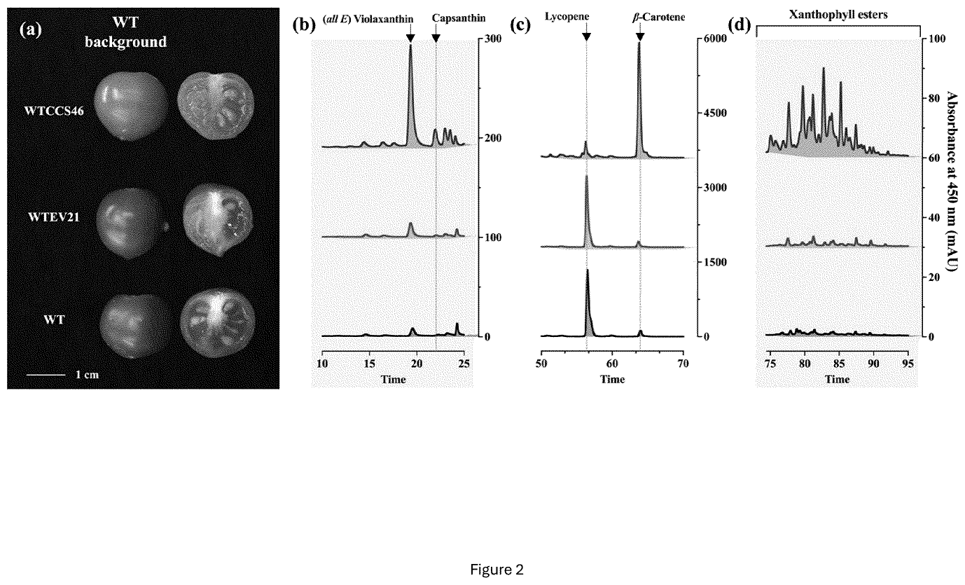

In a control tomato WTEV21 (wildtype background transformed with empty vector, line21), ripe fruit remained red comparable to WT, showing no visible differences generated by the empty vector (FIG. 2a). When tomato was engineered for constitutive expression of pepper CCS, ripe fruit color changed from red to red-orange in the WTCCS46 line (a representative line of wildtype background transformed with CCS, FIG. 2a). The changes in carotenoid profiles behind the fruit color shift were further investigated through HPLC-UV/Vis analysis (FIG. 2b-d). The red fruit from the control lines, WT and WTEV21, contained predominantly lycopene and tiny amounts of other carotenoids, including β-carotene, (all E) violaxanthin, and lutein (FIG. 2b-d) In the ripe fruit of CCS-overexpression lines, capsanthin was identified (FIG. 2b). The primary carotenoid of tomato, lycopene, decreased while β-carotene, (all E) violaxanthin, and xanthophyll esters increased compared to control lines (FIG. 2b-d).

### Example 4: Alteration of Carotenoid Pathway and f-Carotene Enhancement in CCS-Transformed Tomato

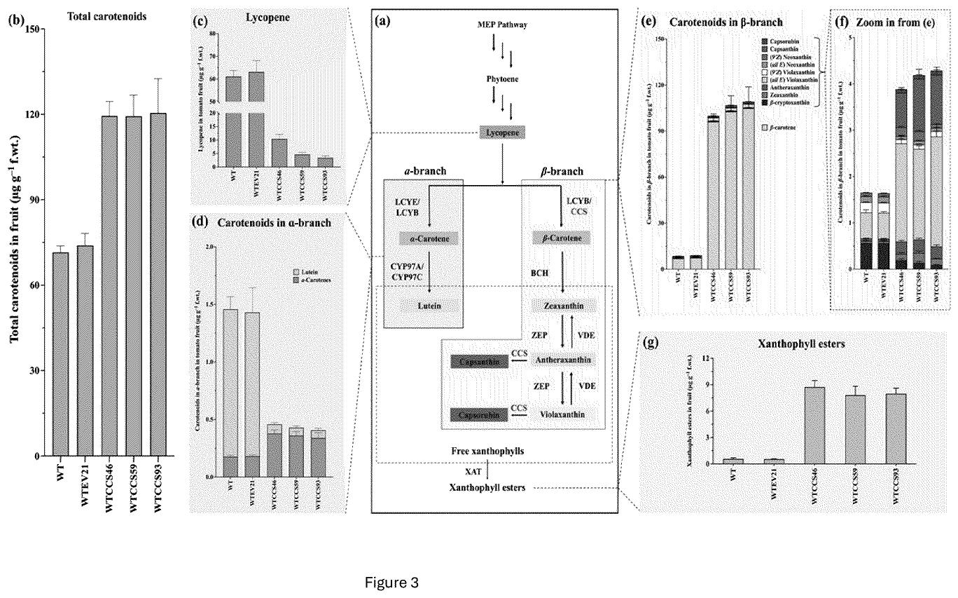

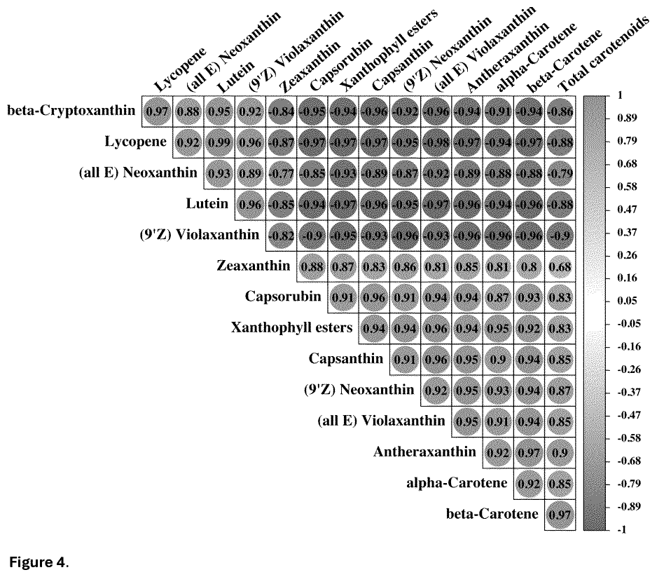

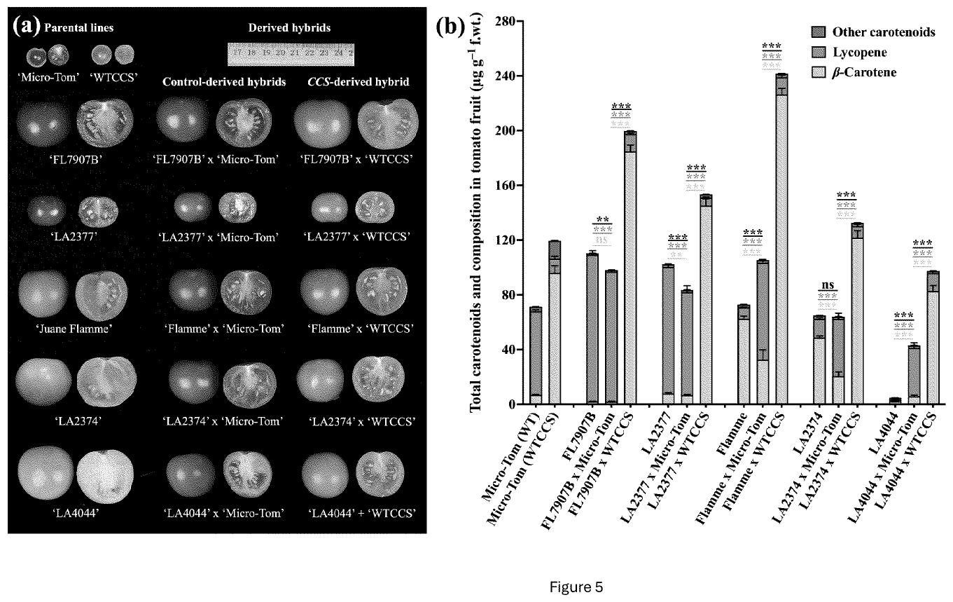

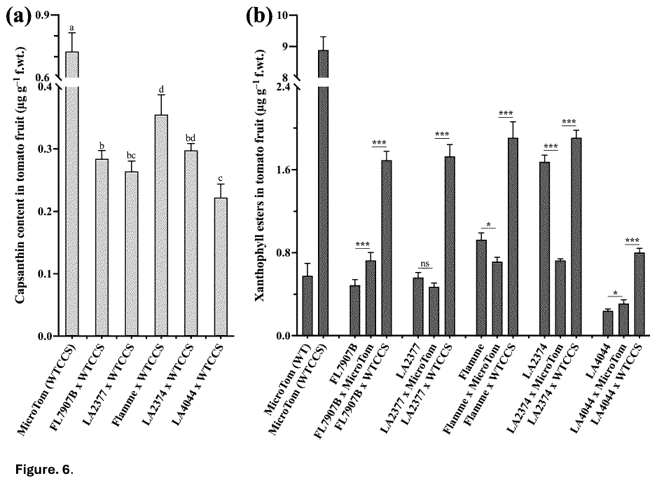

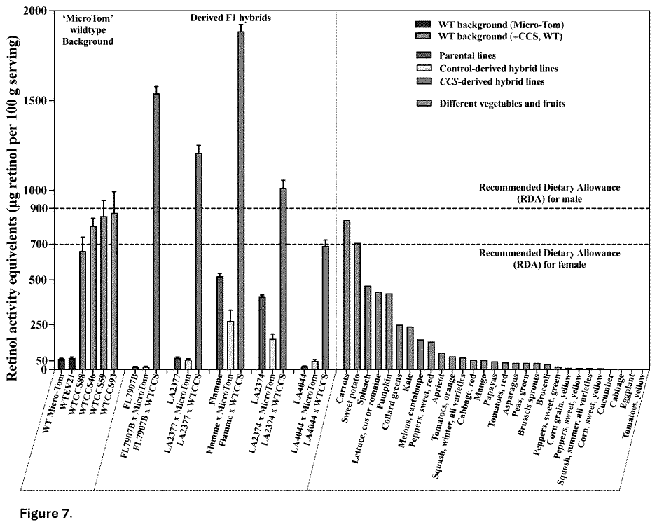

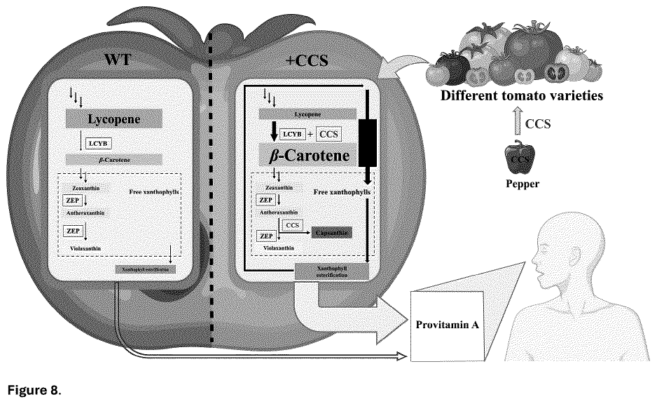

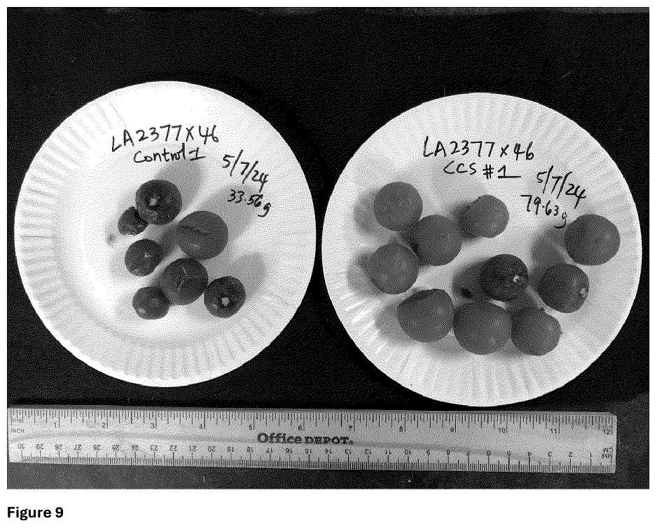

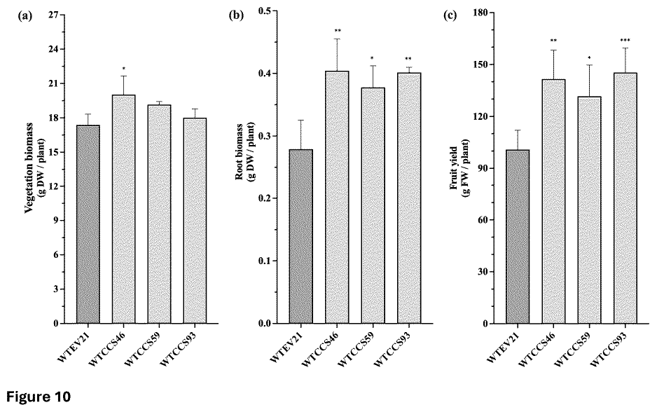

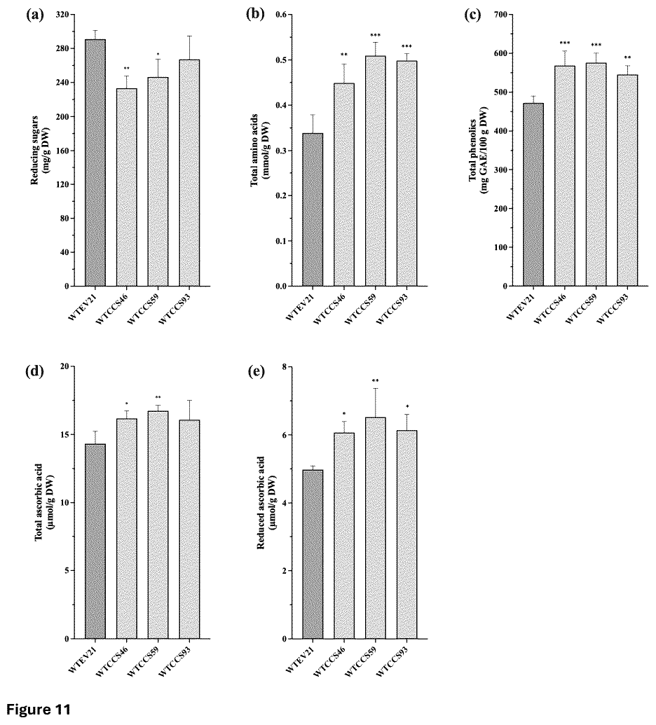

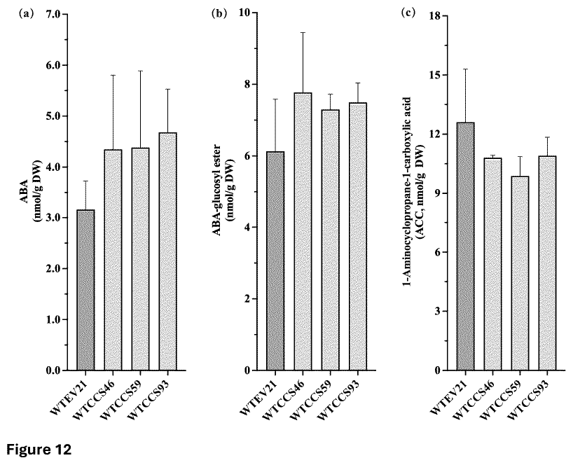

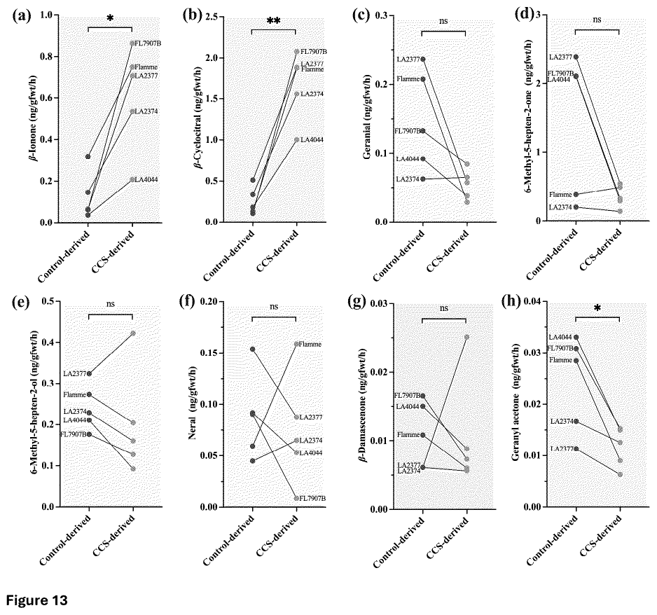

Carotenoid profiles in different tomato lines indicated the alteration of carotenoid compositions (FIG. 2). Specifically, when total carotenoids in tomato fruit were quantified, the fruit of WTCCS lines were all enhanced compared to WT and WTEV21 lines (FIG. 3b). Their fruit had increments of 33-69% more compared to WT and WTEV21 lines. In addition to total carotenoids, the carotenoid compositions were changed in CCS-overexpression lines (FIG. 3c-g). In the red tomato of WT, the carotenoid fraction was mainly lycopene, which is the final carotenoid synthesized before the pathway was split into α- or β-branches (FIG. 3c). Specifically, the amounts of lycopene, in WT and WTEV21 lines, decreased from 85% of total carotenoids to 3-9% in WTCCS lines (FIG. 3c).

Accordingly, other compositions downstream of the carotenoid pathway were altered when CCS was overexpressed in both genetic backgrounds (FIG. 3d-g). Although the amounts of carotenoid in the α-branch were much lower than total carotenoids, lycopene, and β-carotene, a significant decrease of carotenoid contents in the α-branch was observed (FIG. 3d). In α-branch, the amounts of α-carotene increased by 0.9-1.1-fold than the WT and WTEV21 lines (FIG. 3d). However, the lutein content decreased by ˜95% in both genetic backgrounds (FIG. 3d). Opposite to the decrease in total amounts in the α-branch, most compositions, especially β-carotene, in β-branch were increased in CCS-transformed fruit (FIG. 3e, f). Particularly, fruit β-carotene levels enhanced from 7 to 96-105 μg g⁻¹ f.wt. in WTCCS lines (FIG. 3e). Compared to WT and WTEV21 lines, about 11-14-fold increases were observed in WTCCS lines (FIG. 3e). The other carotenoids in the β-branch also showed significant changes in CCS-overexpression lines, although they took minor proportions in this branch (FIG. 3e). Specifically, the amounts of zeaxanthin, antheraxanthin, and (all E) violaxanthin were increased by 1.8-3.9, 3.6-4.5, and 2.5-3.2-fold in WTCCS lines than the WT and WTEV21 lines (FIG. 3f). Since CCS is not native to tomato, no capsanthin or capsorubin was detected in fruit from non-engineered or empty vector-transformed lines (FIG. 3e). However, the fruit of WTCCS lines contained capsanthin at levels of 0.74-1.14 μg g⁻¹ f.wt.. Capsorubin contents in the fruit of WTCCS lines were 0.07 to 0.1 μg g⁻¹ f.wt..

Free xanthophylls generated in both α- and β-branches can be esterified. Ripe fruit of WT and WTEV21 lines accumulated 0.55-0.58 μg g⁻¹ f.wt. xanthophyll esters (FIG. 2d, 3g). In WTCCS lines, ripe fruit contained more xanthophyll esters at levels of 8-10 μg g⁻¹ f.wt., increasing by 12.5-16.8-fold compared to the WT and WTEV21 lines (FIG. 2d, 3g).

The Pearson correlation analysis showed that the carotenoid pools in WTCCS were altered (FIG. 4). Capsanthin and capsorubin contents were positively correlated with the accumulation of α-carotene, β-carotene, antheraxanthin, (all E) violaxanthin, and xanthophyll esters when CCS was overexpressed in both backgrounds. However, the alteration in total carotenoids by overexpressing CCS in the WT background exhibited positive correlations with the amounts of capsanthin, capsorubin, and β-carotene (FIG. 4).

### Example 5: Phenotypic and Carotenoid Compositional Changes in Tomato Fruit of CCS-Derived Lines

To examine the CCS's function in the fruit of some large-fruited tomato of different genetic backgrounds (FIG. 5a), varieties were selected to do controlled crosses with the transgenic line from WTCCS46 line ‘CCS-derived hybrids’ or with WT ‘Micro-Tom’ to generate ‘control-derived hybrids’. Ripe fruit color from the selected maternal lines was red for ‘FL7907B’ and ‘LA2377’, orange for ‘Juane Flamme’ and ‘LA2374’, and yellow for ‘LA4044’ (FIG. 5a). The paternal lines WT ‘Micro-Tom’ and WTCCS (line 46) had red and orange ripe fruit color, and different carotenoid compositions, respectively (FIG. 5a).

After crossing with WT ‘Micro-Tom’, the fruit of F1 hybrid with ‘Juane Flamme’, ‘LA2374’, and ‘LA4044’ turned red to different degrees while F1 hybrids with ‘FL7907B’ and ‘LA2377’ backgrounds remained unchanged (FIG. 5a). Tomato fruit from F1 hybrids of red tomato varieties, ‘FL7907B’ x ‘Micro-Tom’ and ‘LA2377’ x ‘Micro-Tom’, the total carotenoids in the fruit of control-derived lines were decreased by 11-18%, compared to their corresponding maternal lines (FIG. 5b). When using orange and yellow tomato varieties as maternal parents, fruit from F1 hybrids of ‘Juane Flamme’ x ‘Micro-Tom’ and ‘LA4044’ x ‘Micro-Tom’ showed 0.4 and 7.9-fold increase than their corresponding maternal lines respectively (FIG. 5b). No difference was observed between fruits of ‘LA2374’ and ‘LA2374’ x ‘Micro-Tom’ (FIG. 5b). Specifically, the lycopene contents of control-derived hybrids were increased by 2, 7.5 and 28-fold for ‘Juane Flamme’ x ‘Micro-Tom’, ‘LA2374’ x ‘Micro-Tom’, and ‘LA4044’ x ‘Micro-Tom’, but decreased by 0.11 and 0.19-fold for ‘FL7907B’ x ‘Micro-Tom’ and ‘LA2377’ x ‘Micro-Tom’ respectively, compared to their corresponding maternal lines (FIG. 5b). Besides, except for ‘LA4044’, the β-carotene amounts of control-derived hybrids were all decreased by 3-57%, compared to their corresponding maternal lines (FIG. 5b).

When CCS was introduced into the genome of those tomato varieties, all fruit of CCS-derived hybrids from these crosses showed color changing to orange, although their color intensity varied depending on their maternal parents (FIG. 5a). Furthermore, the carotenoid compositions were changed thoroughly compared to control-derived hybrids (FIG. 5b). The total carotenoids of the fruits from CCS-derived hybrids were increased by 0.8-1.3-fold, compared to their corresponding control-derived hybrids (FIG. 5a). Specifically, lycopene amounts were significantly reduced by 63-92%, compared to their corresponding control-derived hybrids (FIG. 5b). The fold changes of β-carotene contents in the fruit from CCS-derived hybrids varied depending on their maternal lines (FIG. 5b). Similar to Micro-Tom transformed with CCS, β-carotene contents in the F1 hybrid tomato fruit generated from red tomato maternal varieties, ‘FL7907B’ x ‘WTCCS’ and ‘LA2377’ x ‘WTCCS’, were increased from 2.1 and 6.7 to 185 and 145 μg g⁻¹ f.wt. respectively, (FIG. 5b). In tomato fruit from F1 hybrids generated from orange tomato maternal varieties, ‘Flamme’ x ‘WTCCS’ and ‘LA2374’ x ‘WTCCS’, β-carotene contents were 226 and 122 μg g⁻¹ f.wt., 2.6 and 1.5-fold more than their corresponding control-derived hybrids (FIG. 5b). F1 hybrid fruit, ‘LA4044’ x ‘WTCCS’, accumulated 5.9 μg g⁻¹ f.wt. β-carotene, 35 times as in its control-derived hybrid (FIG. 5b). Except for ‘LA4044’, four other CCS-derived hybrids showed 0.2-1.4-fold more β-carotene accumulating in fruits than their paternal line, WTCCS46 (FIG. 5b).

In addition to changes in carotenes, capsanthin was detected at levels of 0.22-0.35 μg g⁻¹ f.wt. in the fruit of all CCS-derived hybrids, which were lower than the level of 0.74 μg g⁻¹ f.wt. in the fruit of the paternal line, WTCCS46 (FIG. 6a). Fruit of ‘Flamme’ x ‘WTCCS’ contained the highest capsanthin content among all five F1 hybrids (FIG. 6a). In all fruit from F1 CCS-derived hybrids, the amounts of xanthophyll esters were significantly increased by 0.13-2.5-fold and 1.3-2.7-fold compared to their corresponding maternal and control-derived hybrids respectively (FIG. 6b). Fruit from ‘Flamme’ x ‘WTCCS’ contained the highest xanthophyll ester content among all five F1 hybrids (FIG. 6b).

### Example 6: Retinol Activity Equivalents in Engineered Fruit

To quantify the nutritional improvement of tomato via CCS-overexpression, we evaluated retinol activity equivalents (RAE) computed based on bioactivities of vitamin A and pro-vitamin A (β-carotene, β-cryptoxanthin, and α-carotene) of ripe fruit pericarp from control and CCS-overexpression lines (‘Micro-Tom’ background), as well as from the derived hybrids. No difference in RAE was observed between the fruit of WT and WTEV21 (FIG. 7). Nevertheless, fruits from WTCCS lines contained 662-875 μg retinol activity equivalents per 100 g f.wt. serving, respectively (FIG. 7). The RAE amounts in fruit from CCS-overexpression lines increased by 9.2-13.5 times compared to WT and WTEV21 lines (FIG. 7). Importantly, the RAE amounts from the fruit of most CCS-derived F1 hybrids were observed at significantly higher levels than transgenic ‘Micro-Tom’ overexpressing CCS (WTCCS46, FIG. 7). Specifically, the fruit of CCS-derived F1 hybrids, ‘FL7907B’ x ‘WTCCS’, ‘LA2377’ x ‘WTCCS’, ‘Juane Flamme’ x ‘WTCCS’, ‘LA2374’ x ‘WTCCS’, and ‘LA4044’ x ‘WTCCS’, contained RAE at levels of 1540, 1209, 1885, 1014, and 689 μg per 100 g f.wt. serving, respectively, compared to their corresponding control-derived hybrids at levels of 17, 56, 272, 172, and 49 μg per 100 g f.wt. (FIG. 7).

### Example 7: Postharvest Stability of CCS-Overexpressing and Control Tomato

Fully ripe fruit of ‘Control’ and ‘CCS’ fruit were incubated at 10° C. at 92% humidity in a postharvest incubator for 44 days and the fruit was observed for decay, rot, and mold growth. In six out of seven instances, the fruit of ‘CCS’ expressing lines showed less rotting than the control lines. For this study, CCS expressing MicroTom was crossed to multiple different tomato lines to generate hybrids expressing CCS or vector control to generate control hybrids. Plants were grown in the greenhouse during Fall 2023 and ripe fruit were collected based on color development. Rotting was scored using a scale from 0 to 5, five indicating most rotting and mold growth. Each incubation had 5 to 10 fruit per replicate. The results are shown in Table 4 and illustrated in FIG. 9.

Out of the ten different pairs of lines tested in this experiment, CCS expression decreased the rot score in eight instances (Table 4). Fruit of ‘Flamme’ expressing CCS and ‘Flamme’ control lines did not differ in their rotting scores suggesting that the background carotenoid composition or other factors may operate in determining the impact of the CCS overexpression on fruit rot. Water loss estimated during the incubation period by weighing the fruit on day 1 and day 44, showed that CCS expressing fruit lost less water than their control counterparts in seven out of the ten instances (Table 4). Together these results indicate that CCS overexpression could improve ripe fruit's postharvest stability.

TABLE 4
Postharvest rotting and water loss of fruit from control lines
and CCS-expressing lines of tomato following 44 days of incubation
of ripe fruit at 10° C., 92% RH. The lines that are
indicated as ‘No’ for CCS expression are the control
lines and the ones that are indicated as ‘Yes’ are
expressing CCS. Rot scores are from visual scores using a
scale of 0 to 5, 5 being the highest degree of rotting. Water
loss was computed by changes in fresh weight.

| | | | Water loss |
|---|---|---|---|
| | CCS | Rot score | over 44 days |
| Variety | expression | (scale 0 to 5). | (% of initial wt) |
| ‘MicroTom46’ | No | 5 | 14.9 |
| ‘MicroTomCCS46’ | Yes | 2 | 5.7 |
| ‘LA2377x59’ F1 | No | 4 | 8.6 |
| ‘LA2377xCCS59’ F1 | Yes | 2 | 14.2 |
| ‘LA2377x46’ F1 | No | 5 | 9.5 |
| ‘LA2377xCCS46’ F1 | Yes | 3 | 5.2 |
| ‘AZx46’ F1 | No | 4 | 5.2 |
| ‘AZxCCS46’ F1 | Yes | 2 | 8.3 |
| ‘LA2998x59’ F1 | No | 4 | 15.1 |
| ‘LA2998xCCS59’ F1 | Yes | 3 | 12.5 |
| ‘LA2374x59’ F1 | No | 5 | 14.8 |
| ‘LA2374xCCS59’ F1 | Yes | 4 | 13.7 |
| ‘LA4044x46’ F1 | No | 5 | 16.6 |
| ‘LA4044xCCS46’ F1 | Yes | 4.5 | 11.0 |
| ‘FL7907Bx59’ F1 | No | 5 | 20.7 |
| ‘FL7907BxCCS59’ | Yes | 4 | 13.8 |
| F1 | | | |
| ‘Flammex59’ F1 | No | 4 | 10.1 |
| ‘FlammexCCS59’ F1 | Yes | 4 | 8.22 |
| ‘Flammex46’ | No | 4 | 10.4 |
| FlammexCCS46 | Yes | 4 | 13.4 |

### Discussion Related to Example 1-7

Silencing of CCS in ripe pepper fruit was conducted to test its role in the carotenoid pathway, which has not been investigated until this study (FIG. 1). The capsanthin and capsorubin formation in fruit was abolished when CCS overexpression was knocked down, with a concomitant change in pericarp color to yellow (FIG. 1a), resembling naturally occurring orange and yellow peppers (Ha et al., 2007; Guzman et al., 2010). In pepper fruit, CCS draws the carotenoid pathway to form a new sub-branch to produce ketocarotenoids, capsanthin and capsorubin (FIG. 1e, f). When this sub-β-branch was abolished by VIGS, the flux went downstream of main β-branch to accumulate abundant violaxanthin instead (FIG. 1b, e). However, the flux was not retained in the main β-branch when CCS was silenced. In addition to its function in capsanthin and capsorubin biosynthesis, CCS was reported to have LCYB activity in vitro (Bouvier et al., 1997; Mialoundama et al., 2010). Consistent with this, silencing of CCS reduced, both cyclization of lycopene and formation of ketocarotenoids. Thus, the limited flow into the β-branch caused by the VIGS redirected the flux toward the α-branch, to synthesize α-carotene and lutein (FIG. 1d).

We observed that the amounts of total carotenoids were reduced remarkably in ccs-silenced fruit, suggesting that CCS also assisted in the accumulation of total carotenoids, with more ketocarotenoids produced when CCS functioned normally (FIG. 1c, e). Both ketocarotenoids might be preferable substrates for esterification than lutein and violaxanthin in pepper (FIG. 1c, d, f). This was supported by our results that the reduction of capsanthin, capsorubin, and their derivatives was paralleled by an increase of lutein and violaxanthin in ccs-silenced fruit, while the esterification of lutein and violaxanthin did not complement the amounts of xanthophyll esters (FIG. 2c), reported storage forms of the carotenoid pool (Li et al., 2023). Thus, lack of xanthophyll esterification might cause a flux reduction of the whole carotenoid pathway. Overall, our results elucidated for the first time the critical role of CCS in the metabolic flux and pool of the carotenoid pathway in pepper fruit, although the silencing effect of CCS gene through VIGS has been tested in pepper previously (FIG. 1, Tian et al., 2015).

Given that CCS could change the flux in the carotenoid pathway based on its multiple functions in pepper fruit and has not been used for modifying carotenoid profiles of a ripening fruit yet, its overexpression in the fleshy fruit of tomato allowed us to understand the sink strength in the carotenoid pathway, meanwhile demonstrating the potential applications in crop biofortification. Here, we chose a tomato variety, ‘Micro-Tom’ to overexpress pepper CCS.

In tomato fruit, lycopene is the predominant carotenoid because the expression levels of LCYB are relatively limited, constraining lycopene conversion to β-carotene (Pecker et al., 1996; Ronen et al., 2000; Pandurangaiah et al., 2016). CCS has been suggested to have evolved from LCYB based on analyses of amino acid sequences (Mialoundama et al., 2010), sharing the same conserved motif, FLEET, and lycopene cyclase domain with LCYB. The bottleneck of lycopene cyclization in tomato fruit was alleviated by CCS, attributing to cyclization of more lycopene into β-carotene (FIG. 3c, e). Here, the remarkable enhancement of β-carotene in tomato fruit is comparable or superior to previous studies overexpressing LCYB gene in tomato (Apel and Bock, 2009; D'Ambrosio et al., 2004; Giorio et al., 2008; Mi et al., 2022; Rosati et al., 2000; Zhu et al., 2020). Different from previous studies using LCYB, CCS also functioned in the conversion of antheraxanthin and violaxanthin into capsanthin and capsorubin, respectively (FIG. 2j, k), directing the carotenoid flux moving downstream further (FIG. 3).

Xanthophyll esters in red tomato of WT and WTEV lines represented only a small proportion of carotenoids (FIG. 3g, D'Ambrosio et al., 2011). It differed from pepper since limited free xanthophylls were accumulated in tomato fruit (FIG. 1c, FIG. 3c-f). However, the increased flux in the carotenoid pathway by CCS allowed more xanthophylls to be synthesized downstream than WT and WTEV lines (FIG. 3d-f), which were available for further esterification (FIG. 3g). As a result, the proportion of xanthophyll esters in total carotenoids rose significantly from 0.74-0.81% in WT and WTEV lines to 6.55-9.89% in WTCCS lines (FIG. 3f). Collectively, total carotenoid, β-carotene, and xanthophylls (including zeaxanthin, antheraxanthin, (all E) violaxanthin, capsanthin, and capsorubin) in the β-branch of WTCCS fruit increased along with xanthophyll esters with positive correlation of 0.83, 0.92 and 0.87-0.96, respectively (FIG. 3, FIG. 4a), indicating the positive impact of xanthophyll esterification on carotenoid pools.

Though ‘Micro-Tom’ is a convenient research model, it is dwarf with small fruit (Shikata & Ezura, 2016). Hence, we selected five other large-fruited inbreds with different genetic backgrounds to test CCS overexpression as a strategy to potentially improve their carotenoid profiles. After crossing with ‘Micro-Tom’ WT or ‘WTCCS’, all derived F1 hybrids showed similar plant growth and fruit size to their corresponding maternal lines, indicating that the recessive dwarf allele from ‘Micro-Tom’ had no observable impact on growth and fruiting. However, affected by introducing the genome of ‘Micro-Tom WT’ and ‘WTCCS,’ respectively, the color of fruit from control- and CCS-derived hybrids was altered toward red and orange, respectively, paralleling with the carotenoid compositional changes (FIG. 5). All CCS-derived lines showed significantly higher total carotenoid levels in fruit compared to their corresponding control-derived lines (FIG. 5b). Similar to the observation in WTCCS and 1-1CCS lines (FIG. 3b), lycopene in the fruits of all five CCS-derived varieties was depleted and converted into β-carotene under the catalysis of CCS (FIG. 5b). However, the accumulation of β-carotene in fruit varied from one CCS-derived hybrid to another, among which four showed significantly higher accumulation of β-carotene in fruit than its paternal line, ‘WTCCS’ (FIG. 5b).

In CCS-derived hybrids, ‘FL7907B’ x ‘WTCCS’ and ‘LA2377’ x ‘WTCCS’, the patterns in carotenoid composition were similar to ‘WTCCS’ (FIG. 3, FIG. 5b). CCS overexpression in tomato possibly alleviated the limitation posed by the transcriptional regulation of genes in the carotenoid-associated pathway (Duduit et al., 2022), resulting in a higher metabolic flow moving through lycopene cyclization. Their parental lines, ‘FL7907B’, ‘LA2377’ and ‘Micro-Tom’ WT, had red fruit and accumulated dominantly lycopene but at different levels (FIG. 5b). The fruit of ‘FL7907B’ contained the highest amounts of lycopene among these three parental lines (FIG. 5b). The greater the abundance of lycopene in parental lines, the greater were the amounts of f-carotene in the fruit of CCS-derived hybrids (FIG. 5b). Consequently, ‘FL7907B’ x ‘WTCCS’ accumulated the highest β-carotene contents among WTCCS lines, ‘FL7907B’ x ‘WTCCS’ and ‘LA2377’ x ‘WTCCS’ hybrids (FIG. 5b).

Previously, when LCYB gene was expressed in tomato varieties with red fruit, including ‘Money Maker’ (Rosati et al., 2000; Dharmapuri et al., 2002), ‘Red Setter’ (D'Ambrosio et al., 2004), or ‘Ailsa Craig’ (Zhu et al., 2020), higher accumulation of β-carotene in fruit was observed. However, engineering carotenoid profiles in tomato with yellow or orange fruit is rare. Two varieties, ‘Juane Flamme’ and ‘LA2374’, with orange fruit and higher β-carotene accumulation when crossed to ‘WTCCS’ had even more improvement in β-carotene levels than other parental lines.

The fruit of ‘Juane Flamme’ x ‘WTCCS’ contained the highest β-carotene among all CCS-derived lines, which was near double that of ‘LA2374’ x ‘WTCCS’ (FIG. 5b). It was possibly caused by the difference in promoter region of LCYB (or B allele) between ‘Juane Flamme’ and ‘LA2374’ that caused a higher LCYB expression in ‘Juane Flamme’ (Orchard et al., 2021). The high expression of native LCYB in ‘Juane Flamme’ likely coordinated with CCS to produce more β-carotene than other CCS-derived hybrids (FIG. 5b). Another CCS-derived line, ‘LA4044’ x ‘WTCCS’, also showed a higher accumulation of β-carotene although it was slightly lower than that found in the fruit of ‘WTCCS’ (FIG. 5b). ‘LA4044’ has yellow fruit flesh, in which deletion of 691 bp in the promoter region of PSY gene resulted in its low expression level (Alseekh et al., 2013; Shin et al., 2019). It limited the flux to carotenoids since PSY catalyzes the first committed step in the pathway. Consequently, the fruit of ‘LA4044’ x ‘WTCCS’ accumulated considerably more β-carotene than its control-derived line but lower than the other four CCS-derived hybrids (FIG. 5b).

Like ‘Micro-Tom’ WT transformed with CCS, the synthesis of capsanthin in CCS-derived lines contributed to the increase in esterified xanthophylls (FIG. 6a). The high accumulation of total carotenoids and β-carotene was positively associated with the accumulation of xanthophyll esters (FIG. 3, FIG. 4). Reinforcing this, all five CCS-derived hybrids contained significantly higher amounts of total carotenoids and β-carotene (FIG. 5b), and synergistically accumulated more xanthophyll esters (FIG. 6b), compared to their corresponding control-derived hybrids. For instance, the accumulation of xanthophyll esters in the fruit of ‘LA4044’ x ‘WTCCS’ was lower than the other four CCS-derived lines, attributing to lower total carotenoids and β-carotene than the other four CCS-derived hybrids (FIG. 5, FIG. 6).

Retinol activity equivalent (RAE) is used to evaluate the availability of vitamin A in food (Institute of Medicine (US) Panel on Micronutrients, 2001). Unlike preformed vitamin A from animal-based foods, pro-vitamin A, mainly β-carotene from plant-based foods, is a healthier form for long-term dietary intake since it requires further conversion after uptake and has not been reported to have upper limitations (Blaner, 2020). In addition, the nutritional value is higher in plant-based food in general based on nutrition density scores. To evaluate the nutritional significance of CCS-engineered tomato lines, including ‘Micro-Tom’ and CCS-derived hybrids, we summarized the RAE value from the fruit of these engineered tomato lines along with those from representative vegetables and fruits (FIG. 7).

As a widely consumed vegetable, commercial tomatoes in red, orange, and yellow colors contain limited pro-vitamin A, much lower than many other vegetables and fruits (FIG. 7). CCS-engineered tomato reported here is a significantly better food source than currently available tomatoes, comparable to other high RAE vegetables and fruits or even better (FIG. 7). In particular, the RAE amounts in ripe fruit of WTCCS were comparable to carrots (FIG. 7). Surprisingly, the fruit from CCS-derived hybrid showed even higher RAE values contributed from high β-carotene accumulation (FIG. 5b), among which four of them were higher than male RDA (FIG. 7), indicating the successful biofortification in different commercially viable tomato varieties. In addition to fruits and vegetables, the fruit of CCS-engineered tomato lines, including WTCCS and CCS-derived hybrids, were comparable or even superior to other engineered crop plants in RAE value (Giuliano, 2017). For instance, compared to the first tomato (‘MoneyMaker’) engineered with LCYB gene from Arabidopsis containing ˜458 μg RAE per 100 g serving (Rosati et al., 2000), here, RAE values from the fruit of WTCCS lines and CCS-derived hybrids were significantly improved (FIG. 7). Golden rice contains up to 258 μg RAE per 100 g serving (Paine et al., 2005), which requires 271 and 349 g serving to meet female and male RDA, respectively. In this study, the consumption of 80-106 and 103-136 g of WTCCS fruit, 182-202 and 235-260 g of 1-1CCS fruit, 37-102 and 48-131 g of CCS-derived hybrid fruit meets female and male RDA, respectively (FIG. 7). A demographic survey documented the lack of good vitamin A consumption in 12 East African countries (Wolde & Tessema, 2023). CCS-engineered tomato with high pro-vitamin A is a healthy and safe food source for combating VAD in developing nations. Besides, CCS overexpression in different tomato varieties could provide adequate options for catering to consumers' demands.

In addition to pro-vitamin A, capsanthin and capsorubin are reported to be potentially beneficial for human health (Maoka et al., 2001; Molnir et al., 2005, 2012; Fernández-García et al., 2016; Jo et al., 2017; Joo et al., 2021; Kennedy et al., 2021b; Shanmugham & Subban, 2022). This study is the first report on accumulating capsanthin and capsorubin in a fleshy fruit through metabolic engineering. Previously, ketocarotenoids were reported to accumulate in flower (Viola cornuta L.) and rice endosperm (Ha et al., 2019; Jeong et al., 2021). Pepper CCS, combined with different expression cassettes harboring carotenogenesis gene(s), was introduced into rice to generate grain accumulating up to 0.4 μg/g capsanthin and capsorubin in the endosperm (Ha et al., 2019). By only overexpressing the pepper CCS, we reached comparable results. Ketocarotenoids in WTCCS fruit accumulated at higher levels (FIG. 3f) than in rice grain reported previously, while they were at similar levels in the fruits of all five F1 CCS-derived hybrids (FIG. 6a). Capsanthin has been reported to protect mice against hepatic steatosis and steatohepatitis in nonalcoholic fatty liver disease (Joo et al., 2021). A high fat diet with 0.5 μg g⁻¹ capsanthin, similar to the contents in CCS-engineered fruit, generated a protective effect. (Joo et al., 2021). In summary, constitutive expression of pepper CCS in ‘Micro-Tom’ and selected tomato varieties with different genetic backgrounds altered the metabolic flux in the carotenoid pathway (FIG. 6). The carotenoid flux moved toward β-branch under the function of CCS, producing remarkably higher pro-vitamin A, β-carotene (FIG. 8). Furthermore, considerable amounts of capsanthin and capsorubin accumulated in CCS-engineered tomato (FIG. 8). Along with the production of specialized ketocarotenoids, other xanthophylls of the β-branch were accumulated more in CCS-engineered tomato fruit, contributing to xanthophyll esterification (FIG. 6). Our study demonstrated that CCS could function under different genetic backgrounds, contributing to the accumulation of total carotenoids, β-carotene, and ketocarotenoids in tomato fruit, consequently enhancing its nutritional value (FIG. 7, 8).

CCS overexpression in certain genetic backgrounds increased postharvest stability of ripe tomato (FIG. 9, Table 4). While the mechanisms behind this positive effect of CCS transgene needs further investigation, beta-ionone, a natural breakdown product of beta-carotene has been shown to inhibit Gray mold (Felemban et al., 2024). Beta-ionone levels were significantly increased in CCS-expressing line compared to the fruit of control lines (FIG. 13). Fruit of CCS-overexpressing plants had significantly increased levels of total phenolics, total amino acids, total and reduced ascorbic acid (FIG. 11). Such improvements can be expected to increase the nutritional value and antioxidant qualities of the fruit.

CCS overexpression led to a significant improvement in root biomass, shoot biomass and fruit yield when plants were grown under controlled conditions (FIG. 10). We speculate that modifications in carotenoid composition in tissues led to regulatory changes in the plant to improve both biomass and fruit yield.

### REFERENCES FOR BACKGROUND SECTION AND EXAMPLES 1-7

Alseekh S, Ofner I, Pleban T, Tripodi P, Di Dato F, Cammareri M, Mohammad A, Grandillo S, Fernie A R, Zamir D. 2013. Resolution by recombination: Breaking up Solanum pennellii introgressions. Trends in Plant Science 18: 536-538.
Apel W, Bock R. 2009. Enhancement of carotenoid biosynthesis in transplastomic tomatoes by induced lycopene-to-provitamin a conversion. Plant Physiology 151: 59-66.
Ariizumi T, Kishimoto S, Kakami R, Maoka T, Hirakawa H, Suzuki Y, Ozeki Y, Shirasawa K, Bernillon S, Okabe Y, et al. 2014. Identification of the carotenoid modifying gene PALE YELLOW PETAL 1 as an essential factor in xanthophyll esterification and yellow flower pigmentation in tomato (Solanum lycopersicum). Plant Journal 79: 453-465.
Berry H M, Rickett D V., Baxter C J, Enfissi E M A, Fraser P D. 2019. Carotenoid biosynthesis and sequestration in red chilli pepper fruit and its impact on colour intensity traits. Journal of Experimental Botany 70: 2637-2650.
Blaner W S. 2020. Chapter 5—Vitamin A and provitamin A carotenoids. In: Marriott B P, Birt D F, Stallings V A, Yates A A, eds. Present Knowledge in Nutrition (Eleventh Edition). Academic Press, 73-91.
Bouvier F, D'Harlingue A, Camara B. 1997. Molecular analysis of carotenoid cyclase inhibition. Archives of Biochemistry and Biophysics 346: 53-64.
Bouvier F, Hugueney P, D'Harlingue A, Kuntz M, Camara B. 1994. Xanthophyll biosynthesis in chromoplasts: isolation and molecular cloning of an enzyme catalyzing the conversion of 5,6-epoxycarotenoid into ketocarotenoid. The Plant Journal 6: 45-54.
Butelli E, Titta L, Giorgio M, Mock H P, Matros A, Peterek S, Schijlen E G W M, Hall R D, Bovy A G, Luo J, et al. 2008. Enrichment of tomato fruit with health-promoting anthocyanins by expression of select transcription factors. Nature Biotechnology 26: 1301-1308.
Cheng Y, Pang X, Wan H, Ahammed G J, Yu J, Yao Z, Ruan M, Ye Q, Li Z, Wang R, et al. 2017. Identification of optimal reference genes for normalization of qPCR analysis during pepper fruit development. Frontiers in Plant Science 8: 1-14.
Classic Murashige T, Skoog F. 1962. A revised medium for rapid growth and bioassays with tobacco tissue cultures. Physiol. Plant 15: 473-497.
Cunningham F X, Sun Z, Chamovitz D, Hirschberg J, Gantt E. 1994. Molecular structure and enzymatic function of lycopene cyclase from the cyanobacterium Synechococcus sp strain PCC7942. The Plant Cell 6: 1107-1121.
Cvetković D, Marković D. 2008. UV-effects on antioxidant activity of selected carotenoids in the presence of lecithin estimated by DPPH test. Journal of the Serbian Chemical Society 73: 1051-1061.
D'Ambrosio C, Giorio G, Marino I, Merendino A, Petrozza A, Salfi L, Stigliani A L, Cellini F. 2004. Virtually complete conversion of lycopene into β-carotene in fruits of tomato plants transformed with the tomato lycopene β-cyclase (tlcy-b) cDNA. Plant Science 166: 207-214.
D'Ambrosio C, Stigliani A L, Giorio G. 2011. Overexpression of CrtR-b2 (carotene beta hydroxylase 2) from S. lycopersicum L. differentially affects xanthophyll synthesis and accumulation in transgenic tomato plants. Transgenic Research 20: 47-60.
D'Ambrosio C, Stigliani A L, Rambla J L, Frusciante S, Diretto G, Enfissi E M A, Granell A, Fraser P D, Giorio G. 2023. A xanthophyll-derived apocarotenoid regulates carotenogenesis in tomato chromoplasts. Plant Science 328: 111575.
Delgado-Pelayo R, Hornero-Mendez D. 2012. Identification and quantitative analysis of carotenoids and their esters from sarsaparilla (Smilax aspera L.) berries. Journal of Agricultural and Food Chemistry 60: 8225-8232.
Dharmapuri S, Rosati C, Pallara P, Aquilani R, Bouvier F, Camara B, Giuliano G. 2002. Metabolic engineering of xanthophyll content in tomato fruits. FEBS Letters 519: 30-34.
Duduit J R, Kosentka P Z, Miller M A, Blanco-Ulate B, Lenucci M S, Panthee D R, Perkins-Veazie P, Liu W. 2022. Coordinated transcriptional regulation of the carotenoid biosynthesis contributes to fruit lycopene content in high-lycopene tomato genotypes. Horticulture Research 9: 1-17.
Felemban A, Moreno J C, Mi J, Ali S, Sham A, AbuQamar S F, Al-Babili S. The apocarotenoid β-ionone regulates the transcriptome of Arabidopsis thaliana and increases its resistance against Botrytis cinerea. Plant J. 2024 January; 117(2):541-560. doi: 10.1111/tpj.16510. Epub 2023 Nov. 6. PMID: 37932864.
Fernández-García E, Carvajal-Lérida I, Pérez-Gálvez A. 2016. Carotenoids exclusively synthesized in red pepper (capsanthin and capsorubin) protect human dermal fibroblasts against UVB induced DNA damage. Photochemical and Photobiological Sciences 15: 1204-1211.
Fitzpatrick T B, Basset G J C, Borel P, Carrari F, DellaPenna D, Fraser P D, Hellmann H, Osorio S, Rothan C, Valpuesta V, et al. 2012. Vitamin deficiencies in humans: Can plant science help? Plant Cell 24: 395-414.
Giorio G, Stigliani A L, D'Ambrosio C. 2008. Over-expression of carotene β-hydroxylase 1 (CrtR-b1) and lycopene β-cyclase (Lcy-b) in transgenic tomato fruits. Acta Horticulturae 789: 277-284.
Giuliano G. 2017. Provitamin A biofortification of crop plants: a gold rush with many miners. Current Opinion in Biotechnology 44: 169-180.
Grune T, Lietz G, Palou A, Ross A C, Stahl W, Tang G, Thurnham D, Yin S, Biesalski H K. 2010. β-Carotene Is an Important Vitamin A Source for Humans. The Journal of Nutrition 140: 2268S-2285S.
Guzman I, Hamby S, Romero J, Bosland P W, O'Connell M A. 2010. Variability of carotenoid biosynthesis in orange colored Capsicum spp. Plant Science 179: 49-59.
Ha S H, Kim J K, Jeong Y S, You M K, Lim S H, Kim J K. 2019. Stepwise pathway engineering to the biosynthesis of zeaxanthin, astaxanthin and capsanthin in rice endosperm. Metabolic Engineering 52: 178-189.
Ha S H, Kim J B, Park J S, Lee S W, Cho K J. 2007. A comparison of the carotenoid accumulation in Capsicum varieties that show different ripening colours: Deletion of the capsanthin-capsorubin synthase gene is not a prerequisite for the formation of a yellow pepper. Journal of Experimental Botany 58: 3135-3144.
Institute of Medicine (US) Panel on Micronutrients. 2001. Dietary Reference Intakes for Vitamin A, Vitamin K, Arsenic, Boron, Chromium, Copper; Iodine, Iron, Manganese, Molybdenum, Nickel, Silicon, Vanadium, and Zinc. Washington, D.C.: National Academies Press (US).
Jeong Y S, Kim J K, Baek S A, Lee J Y, Lee D, Ha S H. 2021. Reciprocal Crosses Between Astaxanthin and Capsanthin Rice Unravel Effects of Metabolic Gene Efficacy in Rice Endosperm. Journal of Plant Biology 64: 371-377.
Jo S J, Kim J W, Choi H O, Kim J H, Kim H J, Woo S H, Han B H. 2017. Capsanthin inhibits both adipogenesis in 3T3-L1 preadipocytes and weight gain in high-fat diet-induced obese mice. Biomolecules and Therapeutics 25: 329-336.
Joo H K, Lee Y R, Lee E O, Kim S, Jin H, Kim S, Lim Y P, An C G, Jeon B H. 2021. Protective Role of Dietary Capsanthin in a Mouse Model of Nonalcoholic Fatty Liver Disease. Journal of Medicinal Food 24: 635-644.
Kennedy L E, Abraham A, Kulkarni G, Shettigar N, Dave T, Kulkarni M. 2021a. Capsanthin, a Plant-Derived Xanthophyll: a Review of Pharmacology and Delivery Strategies. AAPS PharmSciTech 22.
Kennedy L E, Abraham A, Kulkarni G, Shettigar N, Dave T, Kulkarni M. 2021b. Capsanthin, a Plant-Derived Xanthophyll: a Review of Pharmacology and Delivery Strategies. AAPS PharmSciTech 22.
Kim S, Ha T Y, Hwang I K. 2009. Analysis, bioavailability, and potential healthy effects of capsanthin, natural red pigment from capsicum spp. Food Reviews International 25: 198-213.
Kim J, Park M, Jeong E S, Lee J M, Choi D. 2017. Harnessing anthocyanin-rich fruit: A visible reporter for tracing virus-induced gene silencing in pepper fruit. Plant Methods 13: 1-10.
Kraemer K, Waelti M, De Pee S, Moench-Pfanner R, Hathcock J N, Bloem M W, Semba R D. 2008. Are low tolerable upper intake levels for vitamin A undermining effective food fortification efforts? Nutrition Reviews 66: 517-525.
Li R, Zeng Q, Zhang X, Jing J, Ge X, Zhao L, Yi B, Tu J, Fu T, Wen J, et al. 2023. Xanthophyll esterases in association with fibrillins control the stable storage of carotenoids in yellow flowers of rapeseed (Brassica juncea). New Phytologist.
Liu Y, Schiff M, Dinesh-Kumar S P. 2002. Virus-induced gene silencing in tomato. The Plant Journal 31: 777-786.
Maoka T, Fujiwara Y, Hashimoto K, Akimoto N. 2001. Capsanthone 3,6-epoxide, a new carotenoid from the fruits of the red paprika Capsicum annuum L. Journal of Agricultural and Food Chemistry 49: 3965-3968.
Maquilan M A D, Padilla D C, Dickson D W, Rathinasabapathi B. 2020. Improved resistance to root-knot nematode species in an advanced inbred line of specialty pepper (Capsicum annuum). HortScience 55: 1105-1110.
Mi J, Vallarino J G, Petrik I, Novik O, Correa S M, Chodasiewicz M, Havaux M, Rodriguez-Concepcion M, Al-Babili S, Fernie A R, et al. 2022. A manipulation of carotenoid metabolism influence biomass partitioning and fitness in tomato. Metabolic Engineering 70: 166-180.
Mialoundama A S, Heintz D, Jadid N, Nkeng P, Rahier A, Deli J, Camara B, Bouvier F. 2010. Characterization of plant carotenoid cyclases as members of the flavoprotein family functioning with no net redox change. Plant Physiology 153: 970-979.
Minguez-Mosquera M I, Hornero-Mendez D. 1993. Separation and Quantification of the Carotenoid Pigments in Red Peppers (Capsicum annuum L.), Paprika, and Oleoresin by Reversed-Phase HPLC. Journal of Agricultural and Food Chemistry 41: 1616-1620.
Molnar P, Kawase M, Satoh K, Sohara Y, Tanaka T, Tani S, Sakagami H, Nakashima H, Motohashi N, Gyémánt N, et al. 2005. Biological activity of carotenoids in red paprika, Valencia orange and Golden delicious apple. Phytotherapy Research 19: 700-707.
Molnar J, Serly J, Pusztai R, Vincze I, Molnar P, Horvath G, Deli J, Maoka T, Zalatnai A, Tokuda H, et al. 2012. Putative supramolecular complexes formed by carotenoids and xanthophylls with ascorbic acid to reverse multidrug resistance in cancer cells. Anticancer Research 32: 507-517.
Nishino A, Yasui H, Maoka T. 2016. Reaction of Paprika Carotenoids, Capsanthin and Capsorubin, with Reactive Oxygen Species. Journal of Agricultural and Food Chemistry 64: 4786-4792.
Orchard C J, Cooperstone J L, Gas-Pascual E, Andrade M C, Abud G, Schwartz S J, Francis D M. 2021. Identification and assessment of alleles in the promoter of the Cyc-B gene that modulate levels of β-carotene in ripe tomato fruit. Plant Genome 14.
Paine J A, Shipton C A, Chaggar S, Howells R M, Kennedy M J, Vernon G, Wright S Y, Hinchliffe E, Adams J L, Silverstone A L, et al. 2005. Improving the nutritional value of Golden Rice through increased pro-vitamin A content. Nature Biotechnology 23: 482-487.
Pandurangaiah S, Ravishankar K V., Shivashankar K S, Sadashiva A T, Pillakenchappa K, Narayanan S K. 2016. Differential expression of carotenoid biosynthetic pathway genes in two contrasting tomato genotypes for lycopene content. Journal of Biosciences 41: 257-264.
Pecker I, Gabbay R, Cunningham F X, Hirschberg J. 1996. Cloning and characterization of the cDNA for lycopene beta-cyclase from tomato reveals decrease in its expression during fruit ripening. Plant Molecular Biology 30: 807-819.
Proost S, Mutwil M. 2018. CoNekT: An open-source framework for comparative genomic and transcriptomic network analyses. Nucleic Acids Research 46: W133-W140.
Ronen G, Carmel-Goren L, Zamir D, Hirschberg J. 2000. An alternative pathway to β-carotene formation in plant chromoplasts discovered by map-based cloning of Beta and old-gold color mutations in tomato. Proceedings of the National Academy of Sciences of the United States of America 97: 11102-11107.
Rosati C, Aquilani R, Dharmapuri S, Pallara P, Marusic C, Tavazza R, Bouvier F, Camara B, Giuliano G. 2000. Metabolic engineering of beta-carotene and lycopene content in tomato fruit. Plant Journal 24: 413-420.
Saito T, Ariizumi T, Okabe Y, Asamizu E, Hiwasa-Tanase K, Fukuda N, Mizoguchi T, Yamazaki Y, Aoki K, Ezura H. 2011. TOMATOMA: A novel tomato mutant database distributing micro-tom mutant collections. Plant and Cell Physiology 52: 283-296.
Shanmugham V, Subban R. 2022. Capsanthin from Capsicum annum fruits exerts anti-glaucoma, antioxidant, anti-inflammatory activity, and corneal pro-inflammatory cytokine gene expression in a benzalkonium chloride-induced rat dry eye model. Journal of Food Biochemistry 46: 1-16.
Shikata M, Ezura H. 2016. Micro-Tom Tomato as an Alternative Plant Model System: Mutant Collection and Efficient Transformation. In: Methods in Molecular Biology. 47-55.
Shin J H, Yoo H J, Yeam I, Lee J M. 2019. Distinguishing two genetic factors that control yellow fruit color in tomato. Horticulture Environment and Biotechnology 60: 59-67.
Van Der Straeten D, Bhullar N K, De Steur H, Gruissem W, MacKenzie D, Pfeiffer W, Qaim M, Slamet-Loedin I, Strobbe S, Tohme J, et al. 2020. Multiplying the efficiency and impact of biofortification through metabolic engineering. Nature Communications 11: 1-10.
Sun H J, Uchii S, Watanabe S, Ezura H. 2006. A highly efficient transformation protocol for Micro-Tom, a model cultivar for tomato functional genomics. Plant and Cell Physiology 47: 426-431.
Tian S L, Li L, Shah S N M, Gong Z H. 2015. The relationship between red fruit colour formation and key genes of capsanthin biosynthesis pathway in Capsicum annuum. Biologia Plantarum 59: 507-513.
Trajković M, Jevremovic S, Dragićević M, Simonović A D, Subotić A R, Milosevi S, Cingel A. 2021. Alteration of flower color in viola Cornuta cv. “lutea splendens” through metabolic engineering of Capsanthin/Capsorubin synthesis. Horticulturae 7.
Wahyuni Y, Ballester A-R, Sudarmonowati E, Bino R J, Bovy A G. 2011. Metabolite biodiversity in pepper (Capsicum) fruits of thirty-two diverse accessions: Variation in health-related compounds and implications for breeding. Phytochemistry 72: 1358-1370.
Wahyuni Y, Ballester A R, Sudarmonowati E, Bino R J, Bovy A G. 2013. Secondary metabolites of Capsicum species and their importance in the human diet. Journal of Natural Products 76: 783-793.
Wolde M, Tessema Z T. 2023. Determinants of good vitamin A consumption in the 12 East Africa Countries using recent Demographic and health survey. PLoS ONE 18: 1-15.
Ye X, Beyer P. 2000. Engineering the provitamin A (3-carotene) biosynthetic pathway into (carotenoid-free) rice endosperm. Science 287: 303-305.
Zheng X, Giuliano G, Al-Babili S. 2020. Carotenoid biofortification in crop plants: citius, altius, fortius. Biochimica et Biophysica Acta—Molecular and Cell Biology of Lipids 1865.
Zhu K, Zheng X, Ye J, Jiang Q, Chen H, Mei X, Wurtzel E T, Deng X. 2020. Building the synthetic biology toolbox with enzyme variants to expand opportunities for biofortification of provitamin a and other health-promoting carotenoids. Journal of Agricultural and Food Chemistry 68: 12048-12057.

### Example 8: CCS Overexpression in Transgenic Tomato Improved Biomass Yield and Fruit Yield

To test whether CCS overexpression would result in an improvement of vegetative growth and fruit yield, three independent CCS-overexpression lines and one vector control in ‘MicroTom’ background were grown under controlled conditions (See methods section below for details). Vegetative biomass remained unchanged in two CCS-transformed lines, while one line, WTCCS46, exhibited a significant increase compared to the control line, WTEV21 (FIG. 10a). Root biomass was significantly higher across all CCS-transformed lines, showing increases of 35.3-44.9% relative to the control line (FIG. 10b). Fruit yield was enhanced in the three CCS-transformed lines, with increases of 40.43%, 30.58%, and 44.08%, respectively, compared to the control yield of 100.86 g fresh fruit per plant (FIG. 10c). These results indicate that expression of CCS in tomato had a significant positive effect on plant growth and fruit yield.

### Methods

Tomato seedlings were grown in half-gallon containers filled with Jolly Gardener C25 potting medium and supplemented with a controlled-release fertilizer (Florikan, N:P:K=18:6:8). Growth conditions included a 16 h light/8 h dark photoperiod, day/night temperatures of 25° C./22° C., 60% relative humidity, and light intensity of approximately 150 μmol m⁻² s⁻¹ provided by full-spectrum LED lighting. Plants received 100 mL of water-soluble fertilizer (MiracleGro Tomato Food, ½ teaspoon per gallon) weekly. Tomatoes were grown until most fruits reached ripening stage, at which point all fruit from each plant were harvested simultaneously. Vegetative tissues (leaf and stem) were also collected at harvest, dried at 75° C., and their dry weights recorded.

### Example 9: CCS-Overexpression Improves Tomato Fruit's Antioxidant and Nutritional Value Via Increasing Free Amino Acids, Total Phenolics and Ascorbic Acid Content

To test whether CCS overexpression could influence metabolite composition of ripe fruit, we tested ripe fruit of control line WTEV21 and those of CCS-overexpression lines WTCCS46, WTCCS59 and WTCCS93 for total reducing sugars, total free amino acids, total phenolics, total and reduced ascorbic acid. These metabolites were chosen for their strong contribution in controlling the fruit's nutritional qualities and taste. In the fruit of CCS-transformed lines, reducing sugar levels decreased by 8.1-19.7% relative to the control, which accumulated 291 mg g⁻¹ DW (FIG. 11a). Despite this reduction, levels of other analyzed metabolites increased significantly (FIG. 11b-e). Compared to the control, total amino acids, total phenolics, total ascorbic acid, and reduced ascorbic acid contents increased by 32.4-46.9%, 15.5-21.9%, 12.2-16.8%, and 21.9-31.0%, respectively (FIG. 11b-e). These results indicated that CCS overexpression could be a tool to improve the antioxidant and nutritional value of tomato via increases in phenolics, amino acids, and ascorbic acid. Our results relating to the reduction of total reducing sugars in the fruit of CCS-overexpression lines suggest feasibility for modifying the sugar content and ‘sugar to acid ratio’ using our genetic engineering strategy.

### Methods

Fully ripe tomato fruit was harvested and flash-frozen using liquid nitrogen. The fruit samples were freeze-dried and powdered prior to metabolite analyses. The reducing sugars were analyzed by a modified colorimetric method (Goncalves, 2010). Each 50 mg freeze-dried sample was extracted with 1 mL of 80% methanol via vortexing, followed by centrifugation at 15000 g for 10 min. The supernatant was collected, and the pellet was re-extracted using the same procedure. Both supernatants were pooled for analysis. For 3,5-dinitrosalicylic acid (DNS) assay, a 250 μL of extract was mixed with 250 μL of DNS reagent in test tubes. Tubes were incubated at 100° C. for 5 min and immediately cooled in ice water while 2.5 mL distilled water was added to stop the reaction. Absorbance was measured at 540 nm. Quantification was performed using a standard curve generated with known concentrations of glucose ranging from 0.05 to 5 mg mL⁻¹. The phenolic content was analyzed using a modified colorimetric method (Ainsworth and Gillespie, 2007). Fifty milligrams of dried tissue was extracted in 300 μL of acidified methanol (1% HCl) overnight at 4° C. The extracts were brought to 200 μL with water, followed by vortexing with 500 μL chloroform and centrifugation at 13000 g for 10 min. The aqueous supernatant was collected. Total phenolics were determined using a modified Folin-Ciocalteu (F-C) microassay. In each reaction, 2.5 μL of sample or gallic acid standard was mixed with 198 μL water and 12.5 μL of Folin-Ciocalteu reagent (Sigma, F9252). After 30 s to 8 min, 37.5 μL of 250 g L⁻¹ sodium carbonate solution was added, and the reaction was incubated at 40° C. for 30 min. Absorbance was read at 765 nm, and concentrations were calculated using a gallic acid standard curve ranging from 0 to 500 mg L⁻¹. Total amino acids were analyzed according to a method established previously with minor modifications (Doi et al., 1981). Briefly, 10 mg dry weight of tissue was mixed with 250 μL of extraction buffer containing 20 mM HEPES (pH 7.0), 5 mM EDTA, and 10 mM NaF, along with 2 mL of chloroform and methanol (2:1, v/v) (Sorrequieta et al., 2010). The homogenate was vortexed thoroughly and incubated on ice for 30 min. The aqueous phases were collected, and amino acids were quantified using a modified Cd-ninhydrin method. For the assay, 0.2 mL of the sample was reacted with 1.5 mL of Cd-ninhydrin reagent, in which 80 mL of 10 g L⁻¹ ninhydrin in absolute ethanol was mixed with 10 mL acetic acid, followed by 1 mL of 1 g mL⁻¹ CdCl₂. The mixture was incubated at 84° C. for 10 min. After cooling, absorbance was measured at 505 nm.

Ascorbic acid levels were analyzed using the methods of Stevens et al. (2008) with the modifications of Di Matteo et al. (2010). One hundred milligrams of dried powder was homogenized in 600 μL of ice-cold 6% (wt/v) trichloroacetic acid. The mixture was vortexed for 10 s, followed by centrifugation at 25000 g for 15 min at 4° C. A 20 μL aliquot of the resulting supernatant was used for colorimetric assays. Two assays were performed per sample to quantify total and reduced ascorbate content. For total ascorbate, 5 mM DTT, the reducing agent was added, whereas reduced ascorbate was measured without DTT. In each assay, 20 μL of sample or standard was pipetted into a 96-well microplate, followed by 20 μL of either 5 mM DTT (total) or 0.4 M phosphate buffer (pH 7.4, reduced). After incubation at 37° C. for 20 min, 10 μL of either 0.5% N-ethyl maleimide (total) or phosphate buffer (reduced) was added, along with 80 μL of color reagent. Plates were incubated again at 37° C. for 40 min, and absorbance was read at 550 nm. Ascorbate concentrations were determined using a standard curve.

### REFERENCES FOR EXAMPLE 9

Ainsworth, E. A., & Gillespie, K. M. (2007). Estimation of total phenolic content and other oxidation substrates in plant tissues using Folin-Ciocalteu reagent. Nature protocols, 2(4), 875-877.
Boches, P., Peterschmidt, B., & Myers, J. R. (2011). Evaluation of a subset of the Solanum lycopersicum var. cerasiforme core collection for horticultural quality and fruit phenolic content. HortScience, 46(11), 1450-1455.
Di Matteo, A., Sacco, A., Anacleria, M., Pezzotti, M., Delledonne, M., Ferrarini, A., & Barone, A. (2010). The ascorbic acid content of tomato fruits is associated with the expression of genes involved in pectin degradation. BMC plant biology, 10, 1-11.
Doi, E., Shibata, D., & Matoba, T. (1981). Modified colorimetric ninhydrin methods for peptidase assay. Analytical Biochemistry, 118(1), 173-184.
Gonealves, C., Rodriguez-Jasso, R. M., Gomes, N., Teixeira, J. A., & Belo, I. (2010). Adaptation of dinitrosalicylic acid method to microtiter plates. Analytical Methods, 2(12), 2046-2048.
Sorrequieta, A., Ferraro, G., Boggio, S. B., & Valle, E. M. (2010). Free amino acid production during tomato fruit ripening: a focus on L-glutamate. Amino acids, 38, 1523-1532.
Stevens, R., Page, D., Gouble, B., Garchery, C., Zamir, D., & Causse, M. (2008). Tomato fruit ascorbic acid content is linked with monodehydroascorbate reductase activity and tolerance to chilling stress. Plant, cell & environment, 31(8), 1086-1096.

### Example 10: CCS Overexpression has Little Impact on the Levels of Phytohormone ABA and Phytohormone Precursor ACC in the Fruit

Since the synthetic pathway to the stress hormone ABA is connected to the synthesis of carotenoids, we tested the levels of ABA in ripe fruit from control and CCS-overexpression lines. We also measured the precursor of ethylene, the phytohormone having a central role in fruit ripening. In CCS-transformed fruit, ABA contents ranged from 4.35 to 4.68 nmol g⁻¹ DW, showing an increasing trend compared to the control (3.16 nmol g⁻¹ DW), though the differences were not statistically significant. Similarly, the levels of ABA-glycosyl ester, the storage form of ABA, increased from 6.13 nmol g⁻¹ DW in the control to 7.30-7.77 nmol g⁻¹ DW in transformed lines. However, other ABA conjugates were below the detection limit. In contrast, the levels of 1-aminocyclopropane-1-carboxylic acid (ACC), the immediate precursor of ethylene, decreased from 12.61 nmol g⁻¹ DW in the control to 9.88-10.92 nmol g⁻¹ DW in fruit from CCS-transformed lines (FIG. 12). Our results suggest that CCS-overexpression did not alter ABA and ACC levels drastically enough to alter additional traits of the plant or fruit quality.

### Methods

Fully ripe tomato fruit was harvested and flash frozen using liquid nitrogen, freeze-dried and powdered. One milliliter of methanol/water/formic acid (15:4:1, V/V/V) with 10 μL of internal standard (100 ng/mL) was added to 15 mg of dried fruit tissue. The mixture was vortexed for 10 minutes, followed by centrifugation at 12000 g at 4° C. for 5 min. The supernatant was transferred to a clean plastic microtube and dried by evaporation. The samples were reconstituted in 100 μL of 80% methanol (V/V) and filtered through a 0.22-μm filter. Two microliters of processed extracts were injected into an UPLC-ESI-MS/MS system (UPLC, ExionLC™ AD; ESI-MS/MS, Applied Biosystems 6500 Triple Quadrupole) for analysis (Pan et al., 2020). The chromatogram separation was achieved through a C18 column (100 mm×2.1 mm i.d., 1.8 μm, Waters ACQUITY UPLC HSS T3) and a solvent system with two components namely water (A) and acetonitrile (B) both with 0.04% acetic acid. The gradient program was: 95% A at 1 min, gradually reaching 5% A at 8 min, hold for 1 min, then return to 95% A at 9 min followed by ahold for another 3 min. The flow rate was at 0.35 mL/min. Phytohormones including ABA, ABA-glycosyl ester, abscisic aldehyde, ACC, 5-deoxystrigol, strigol were qualified and quantified against a mixture of standards ranging from 0.01 to 500 ng mL⁻¹. Abscisic aldehyde, 5-deoxystrigol, and strigol were under the lower limit of quantification of 1.0, 0.01, 0.5 ng/mL respectively, indicating the amounts of three compounds were lower than 6.67, 0.067, 3.33 ng per gram of fruit dried weight.

### REFERENCES FOR EXAMPLE 10

Pan, X., Welti, R., & Wang, X. (2010). Quantitative analysis of major plant hormones in crude plant extracts by high-performance liquid chromatography-mass spectrometry. Nature protocols, 5(6), 986-992.

### Example 11: CCS Overexpression has Increased Fruit Volatile Organic Compounds Generated from Beta-Carotene and Decreased Volatile Organic Compounds Generated from Lycopene

Certain volatile organic compounds in fruit including certain aromatic ones are known to be generated by oxidative breakdown of carotenoids. We tested the hypothesis that increased beta-carotene levels in CCS-expressing fruit could have significantly altered the levels of volatiles of the class apocarotenoids. For this purpose, to precisely study the impact of CCS transgenic expression in the background of different tomato varieties, plants expressing CCS (in the MicroTom background) was crossed to different tomato varieties known to have fruit of varying fruit qualities and F1 hybrids were derived. Ripe fruit from plants expressing transgenic CCS and the negative control (vector control MicroTom background crossed to the test tomato variety) were grown in a greenhouse and their ripe fruit were analyzed. Volatile organic compounds were quantified using GC-mass spectrometry (FIG. 13).

In control-derived hybrids, ‘Flamme’ x ‘Micro-Tom’ and ‘LA2374’ x ‘Micro-Tom’ with orange fruit as one of the original parents showed higher amounts of β-ionone and β-cyclocitral, compared to the other control-derived hybrids (FIG. 13a&b). When CCS was overexpressed, both β-ionone and β-cyclocitral amounts were increased by 1.3 to 13.0-fold and 2.7 to 19.1-fold in all five CCS-derived hybrids (FIG. 13a&b). The amounts of geranial, geranyl acetone, 6-methyl-5-hepten-2-one, and 6-methyl-5-hepten-2-ol showed a decreasing trend in CCS-derived hybrids with one exception for ‘LA2377’ x ‘WTCCS’ and the decrease in geranyl acetone was statistically significant (FIG. 13c-d).

### Methods

Freshly harvested ripe fruit from greenhouse-grown control and CCS-derived hybrids were used to quantify volatile apocarotenoids (Tieman et al., 2006). Briefly, a 100 g sample of chopped fruit was placed inside a glass tube through which air filtered through a hydrocarbon trap (Agilent, Palo Alto, CA) was flowing through for 1 h. The volatile apocarotenoids were collected on a Super Q column, which was then eluted with methylene chloride after the addition of nonyl acetate as an internal standard. VOC were separated on an Agilent (Palo Alto, CA) DB-5 column and analyzed using an Agilent 6890N gas chromatograph. Compounds were identified based on retention times compared to standard compounds (Sigma Aldrich, St Louis, MO). The volatile amounts were computed based on peak areas calibrated by internal standards and expressed as ng⁻¹ gfwt⁻¹ h⁻¹.

### REFERENCE

Tieman D M, Zeigler M, Schmelz E A, Taylor M G, Bliss P, Kirst M, Klee H J. 2006. Identification of loci affecting flavour volatile emissions in tomato fruits. Journal of Experimental Botany 57: 887-896.

What is claimed is:

1. A method of increasing carotenoid content in a tomato plant compared to a wild type tomato plant, the method comprising producing a transgenic plant comprising a recombinant DNA construct that comprises a polynucleotide sequence comprising SEQ ID NO: 1, or SEQ ID NO: 2, or a variant thereof.

2. The method according to claim 1, wherein the recombinant DNA construct comprises a promoter functional in a plant cell and operably-linked to the polynucleotide sequence.

3. The method according to claim 1, wherein the plant is produced by transforming a plant cell or tissue with the recombinant DNA construct, and regenerating or developing the transgenic plant from the plant cell or tissue comprising the recombinant DNA construct.

4. The method according to claim 1, comprising: producing a progeny plant comprising the recombinant DNA construct by crossing the transgenic plant with: a) itself; b) a second plant from the same plant line; c) a plant of a different species; or d) a second plant from a different plant line, to produce a seed, growing the seed to produce a progeny plant; and selecting a progeny plant with increased carotenoid content as compared to a control plant.

5. The method according to claim 1, wherein the transgenic plant is produced by integration of the recombinant DNA construct into the genome of a plant cell or tissue using a donor template comprising the recombinant DNA construct, and regenerating or developing the transgenic plant from the plant cell or tissue comprising the recombinant DNA construct.

6. A transgenic plant produced by the method of claim 1.

7. A recombinant DNA construct that comprises a polynucleotide sequence comprising SEQ ID NO: 1 or SEQ ID NO: 2, or a variant thereof.

8. The recombinant DNA construct of claim 7 comprising a promoter functional in a plant cell and operably linked to the polynucleotide sequence.

9. A vector or plasmid comprising the recombinant DNA construct of claim 7.

10. A plant comprising the recombinant DNA construct of claim 7, wherein the plant is optionally tomato.

11. A commodity product made from the plant of claim 6, wherein the plant comprises fruit comprising enhanced levels of beta-carotene, capsanthin and/or capsorubin compared to fruit from a wild-type plant.

12. A commodity product made from the plant of claim 6, where in the plant comprises fruit comprising enhanced postharvest stability and resistance to rotting as compared to fruit from a wild-type plant.

13. A commodity product made from the plant of claim 6, where in the plant comprises fruit comprising enhanced levels of ascorbic acid as compared to fruit from a wild-type plant.

14. The commodity product of claim 11, wherein the commodity product comprises whole fruit or fruit juice.

15. A plant of claim 6 wherein the plant comprises enhanced fruit yield as compared to a wild-type plant.

16. A plant of claim 6 wherein the plant comprises fruit with quantitatively and qualitatively modified levels of carotenoid-derived volatiles compared to fruit of a wild-type plant.
